(19) United States
(12) Patent Application Publication
(10) Pub. No.: US 2022/0282282 A1
(43) Pub. Date: Sep. 8, 2022

(54) BUFFER SOLUTIONS FOR ELECTROPORATION

(71) Applicant: Precigen, Inc., Germantown, MD (US)

(72) Inventors: Bryan BUTMAN, Germantown, MD (US); Robert Daniel SLONE, Germantown, MD (US); Steven ROBERTS, Germantown, MD (US); Chad B. GREEN, Campbell, CA (US); Vincent SO, Campbell, CA (US)

(21) Appl. No.: 17/682,793

(22) Filed: Feb. 28, 2022

Related U.S. Application Data
(60) Provisional application No. 63/154,050, filed on Feb. 26, 2021.
(60) Provisional application No. 63/184,908, filed on May 6, 2021.

(51) Int. Cl. C12N 15/87; A61K 9/08; A61K 47/26; A61K 47/20; A61K 47/10; A61K 47/36

(52) U.S. Cl. CPC C12N 15/87; A61K 9/08; A61K 47/26; A61K 47/20; A61K 47/10; A61K 47/36

(57) ABSTRACT

An electroporation buffer comprising: a solvent; a sugar; a chloride salt; and a buffering agent. In certain embodiments: the solvent is water; the sugar is glucose or mannitol; the chloride salt is potassium chloride (KCl) or magnesium chloride (MgCl₂); and the buffering agent is sodium phosphate, 4-(2-hydroxyethyl)-1-piperazineethanesulfonic acid (HEPES) and/or dimethyl sulfoxide (DMSO). A method of electroporation, the method comprising applying an electric current to a suspension comprising: isolated eukaryotic cells; a biological material that is exogenous to the cells; and the aforementioned buffer. A recombinant cell produced using such a method. An electroporation apparatus comprising: one or more chambers; one or more pairs of electrodes configured to generate electric fields within the one or more chambers, wherein each electric field corresponds to one chamber; and a flow channel. A method for electroporation comprising utilizing the aforementioned electroporation apparatus.

### FIELD OF THE INVENTION

The invention relates to buffers capable of delivering biologically active material into cells using electric current, methods for introducing biologically active material into cells using the buffers, and a kit comprising the buffer.

### INCORPORATION BY REFERENCE

All publications, patents, and patent applications herein are incorporated by reference in their entireties to the same extent as if each individual publication, patent, or patent application was specifically and individually indicated to be incorporated by reference in its entirety. In the event of a conflict between a term defined herein and a term in an incorporated reference, the term defined herein controls.

### BACKGROUND OF THE INVENTION

A primary method for introducing exogenous biological material into cells is electroporation (EP). This method is commonly used for the genetic engineering, modification or other manipulation of cell properties and functions. During electroporation, the biological material is dissolved in a buffer solution and then introduced into the cell utilizing an electric current. In this process, the cell membrane is made permeable by the action of short electrical pulses, thus allowing the biologically active material to enter the cell, a process known as “transfection.”

During electroporation, the efficiency by which the biological materials are transfected into the cells and/or the subsequent viability of the transfected cells may be undesirably low. This low transfection efficiency and/or low post-electroporation viability may be due to several factors, including: electroporation conditions (e.g., applied voltage, duration and nature of cell handling); the composition of buffer used; the nature of biological material being introduced; the health, age and inherent viability of the host cell population; cell type; cell density (in solution); cell confluency; cell division rate, cell fragility, cell cycle phase (e.g., growing, dividing, or quiescent cells); cell sensitivity to contact inhibition; and, the like.

Many electroporation buffers provide less than desirable transfection efficiencies and/or contain an undesirably high number of chemical components, some of which can be harmful to cell viability or to transfection efficiency. Accordingly, there is a need in the art for simplified and/or optimized electroporation buffers, i.e., buffers containing fewer overall components, yet still capable of achieving high transfection efficiencies and correspondingly high transfected cell viability rates.

### SUMMARY OF THE INVENTION

Provided herein is an electroporation (EP) buffer that comprises fewer chemical components than many electroporation buffers on the market, and yet is capable of achieving surprisingly high transfection efficiency and post-electroporation cell viability rates. The buffer accomplishes this by providing a synergistic combination and concentration of chemical components that are optimized to maximize the uptake of biological material into cells while minimizing any harm to the cells.

In some embodiments, the buffer is capable of transfecting a population of cells by electroporation. In certain embodiments, the cells are eukaryotic cells. In certain embodiments, the cells are mammalian cells. In certain embodiments, the cells are human cells. In certain embodiments, the cells are immune cells, for example, neutrophils, eosinophils, basophils, mast cells, monocytes, macrophages, dendritic cells, natural killer cells, and/or lymphocytes (e.g., B cells and T cells).

The present invention thus relates in part to a buffer that comprises a solvent, a sugar, one or more chloride salts, and one or more buffering agents. In some such embodiments: the solvent is water; the sugar is glucose or mannitol; the one or more chloride salts comprises potassium chloride (KCl) and/or magnesium chloride (MgCl₂); and/or the one or more buffering agents comprises 4-(2-hydroxyethyl)-1-piperazineethanesulfonic acid (HEPES), dimethyl sulfoxide (DMSO), Na₂HPO₄ (dibasic sodium phosphate), NaH₂PO₄ (monobasic sodium phosphate), and/or a combination of Na₂HPO₄ and NaH₂PO₄ (referred to herein interchangeably as Na₂HPO₄/NaH₂PO₄ or sodium phosphate). In certain embodiments, the buffer does not comprise DMSO and/or HEPES.

In some embodiments, the buffer comprises, consists essentially of, or consists of: water; glucose and/or mannitol; KCl and/or MgCl₂; and sodium phosphate. In certain embodiments, the buffer comprises, consists essentially of, or consists of: water; glucose and/or mannitol; KCl and/or MgCl₂; sodium phosphate; and HEPES and/or DMSO.

In some embodiments, the glucose in the buffer is present in an amount of from about 10 mM to about 50 mM, from about 10 mM to about 40 mM, from about 10 mM to about 20 mM, or from about 25 mM to about 35 mM. In certain embodiments, glucose is present in an amount of from about 25 mM to about 35 mM. In certain embodiments, glucose is present in an amount of about 30 mM or about 31 mM.

In some embodiments, the mannitol in the buffer is present in an amount of from about 10 mM to about 50 mM, from about 10 mM to about 40 mM, from about 10 mM to about 20 mM, or from about 25 mM to about 35 mM. In certain embodiments, mannitol is present in an amount of from about 25 mM to about 35 mM. In certain embodiments, mannitol is present in an amount of about 30 mM or about 31 mM.

In some embodiments, the KCl in the buffer is present in an amount of from about 1 mM to about 30 mM, from about 2 mM to about 25 mM, from about 3 mM to about 20 mM, from about 4 mM to about 15 mM, from about 5 mM to about 15 mM, or from about 5 mM to about 10 mM. In certain embodiments, KCl is present in an amount of from about 5 mM to about 15 mM. In certain embodiments, KCl is present in an amount of about 5 mM or about 10 mM.

In some embodiments, the MgCl₂ in the buffer is present in an amount of from about 5 mM to about 50 mM, from about 6 mM to about 45 mM, from about 7 mM to about 40 mM, from about 8 mM to about 35 mM, from about 9 mM to about 30 mM, from about 10 mM to about 25 mM, or from about 15 mM to about 25 mM. In certain embodiments, MgCl₂ is present in an amount of about 10 mM or about 15 mM.

In some embodiments, the sodium phosphate in the buffer is present in an amount of from about 50 mM to about 160 mM, from about 60 mM to about 150 mM, from about 70 mM to about 140 mM, from about 75 mM to about 130 mM, from about 80 mM to about 125 mM, from about 90 mM to about 125 mM, or from about 90 mM to about 120 mM. In certain embodiments, sodium phosphate is present in an amount of about 90 mM to about 120 mM. In certain embodiments, sodium phosphate is present in an amount of about 90 mM or about 105 mM.

In some embodiments, the HEPES in the buffer is present in an amount of from about 1 mM to about 30 mM, from about 2 mM to about 25 mM, from about 3 mM to about 20 mM, from about 4 mM to about 15 mM, or from about 5 mM to about 10 mM. In certain embodiments, HEPES is present in an amount of about 5 mM to about 10 mM. In certain embodiments, HEPES is present in an amount of about 5 mM or about 10 mM.

In some embodiments, the DMSO in the buffer is present in an amount of from 0% to about 2.5%, from about 0.1% to about 5%, from about 1% to about 5%, from about 2% to about 5%, from about 3% to about 5%, or from about 4% to about 5% by volume of the total buffer volume.

In some embodiments, the buffer comprises, consists essentially of, or consists of: water; glucose and/or mannitol in an amount of from about 25 mM to about 35 mM; KCl in an amount of from about 5 mM to about 15 mM; MgCl₂ in an amount of from about 15 mM to about 25 mM; and sodium phosphate in an amount of from about 90 mM to about 120 mM. In certain embodiments, the buffer comprises, consists essentially of, or consists of: water; glucose and/or mannitol in an amount of from about 25 mM to about 35 mM; KCl in an amount of from about 5 mM to about 15 mM; MgCl₂ in an amount of from about 15 mM to about 25 mM; sodium phosphate in an amount of from about 90 mM to about 120 mM; and HEPES in an amount of 0 mM to about 10 mM or from about 5 mM to about 10 mM and/or DMSO in an amount equal to or less than about 2.5% by volume of the total volume of the buffer.

In some embodiments, the buffer comprises, consists essentially of, or consists of: water; about 30 mM glucose or mannitol; about 10 mM KCl; about 20 mM MgCl₂; about 105 mM of sodium phosphate; and about 5 mM of HEPES. In certain embodiments, the buffer comprises, consists essentially of, or consists of: water; about 30 mM glucose or mannitol; about 10 mM KCl; about 20 mM MgCl₂; about 105 mM of sodium phosphate; about 5 mM of HEPES; and DMSO in an amount equal to or less than about 2.5% by volume of the total volume of the buffer.

In some embodiments, the buffer comprises, consists essentially of, or consists of: water; about 31 mM glucose or mannitol; about 5 mM KCl; about 15 mM MgCl₂; and about 90 mM of sodium phosphate. In certain embodiments, the buffer comprises, consists essentially of, or consists of: water; about 31 mM glucose or mannitol; about 5 mM KCl; about 15 mM MgCl₂; and about 90 mM of sodium phosphate; and HEPES in an amount of from about 5 mM to about 10 mM and/or DMSO in an amount equal to or less than about 2.5% by volume of the total volume of the buffer.

In some embodiments, the buffer comprises, consists essentially of, or consists of: water; about 30 mM glucose or mannitol; about 5 mM KCl; about 15 mM MgCl₂; about 90 mM of sodium phosphate; and about 10 mM of HEPES. In certain embodiments, the buffer comprises, consists essentially of, or consists of: water; about 30 mM glucose or mannitol; about 5 mM KCl; about 15 mM MgCl₂; about 90 mM of sodium phosphate; about 10 mM of HEPES; and DMSO in an amount equal to or less than about 2.5% by volume of the total volume of the buffer.

In some embodiments, the buffer has an osmolality lower than intracellular osmolality. In certain such embodiments, the osmolality may be from about 275 mOsm/kg H₂O to about 350 mOsm/kg H₂O.

In some embodiments, the buffer has a conductivity of about 10.0 ms/cm to about 15.0 ms/cm.

In some embodiments, the buffer has a pH of about 7.0 to about 7.1.

The present invention also relates in part to a method of electroporation comprising: applying an electric current to a suspension comprising isolated eukaryotic cells; a biological material that is exogenous to the cells; and the buffer of the present invention. The application of the electric current to the suspension facilitates the introduction of the biological material into the cells. In some embodiments, the eukaryotic cells are human cells. In certain embodiments, the biological material comprises a nucleic acid, a polypeptide, a peptide, and/or a ribonucleoprotein.

In some embodiments, at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, at least 2×10⁹ cells, at least 3×10⁹ cells, at least 4×10⁹ cells, at least 5×10⁹ cells, at least 6×10⁹ cells, at least 7×10⁹ cells, at least 8×10⁹ cells, at least 9×10⁹ cells, at least 1×10¹⁰ cells, at least 2×10¹⁰ cells, at least 3×10¹⁰ cells, at least 4×10¹⁰ cells, at least 5×10¹⁰ cells, at least 6×10¹⁰ cells, at least 7×10¹⁰ cells, at least 8×10¹⁰ cells, at least 9×10¹⁰ cells, at least 1×10¹¹ cells, at least 2×10¹¹ cells, at least 3×10¹¹ cells, at least 4×10¹¹ cells, at least 5×10¹¹ cells, at least 6×10¹¹ cells, at least 7×10¹¹ cells, at least 8×10¹¹ cells, at least 9×10¹¹ cells, at least 1×10¹² cells, at least 2×10¹² cells, at least 3×10¹² cells, at least 4×10¹² cells, at least 5×10¹² cells, at least 6×10¹² cells, at least 7×10¹² cells, at least 8×10¹² cells, or at least 9×10¹² are involved in the electroporation process.

The present invention also relates in part to a method of increasing transfection efficiency, the method comprising: combining insolated eukaryotic cells and a biological material that is exogenous to the cells with the buffer of the present invention, thereby forming a suspension; and applying an electric current to the suspension, thereby facilitating the introduction of the biological material into the cells. In some embodiments, the transfection efficiency when such method is used is measured to be at least 1.35 times higher than when a method using a control buffer is used. In certain embodiments, the biological material comprises a nucleic acid, a polypeptide, a peptide, and/or a ribonucleoprotein. In certain embodiments, the cells are lymphocytes, for example T cells.

The present invention also relates in part to a method of increasing the recovery of transfected cells, the method comprising: combining insolated eukaryotic cells and a biological material that is exogenous to the cells with the buffer of the present invention, thereby forming a suspension; and applying an electric current to the suspension, thereby facilitating the introduction of the biological material into the cells. In some embodiments, the recovery of transfected cells when such method is used is measured to be at least 1.53 times higher than when a method using a control buffer is used. In certain embodiments, the biological material comprises a nucleic acid, a polypeptide, a peptide, and/or a ribonucleoprotein. In certain embodiments, the cells are lymphocytes, for example T cells.

The present invention also relates in part to a recombinant cell produced using a method that utilizes the buffer of the present invention. In some embodiments, the cell is a recombinant human immune cell. In certain embodiments, the cell is a recombinant lymphocyte. In certain embodiments, the cell is a recombinant T-cell.

The present invention also relates in part to a method of immunotherapy using a recombinant T-cell that has been produced using a method that utilizes the buffer of the present invention.

The present invention also relates in part to a method of immunotherapy using a chimeric antigen receptor (CAR) T-cell that has been produced using a method that utilizes the buffer of the present invention.

The present invention also relates in part to the use of a recombinant T-cell that has been produced using a method that utilizes the buffer of the present invention in the preparation of a medicament for the treatment of a disease or disorder.

The present invention also relates in part to the use of a CAR T-cell that has been produced using a method that utilizes the buffer of the present invention in the preparation of a medicament for the treatment of a disease or disorder.

The present invention also relates in part to a kit for use in electroporation. In some embodiments, the kit comprises: a buffer of the present invention; and a dropper, pipette, or cuvette. In certain embodiments, the kit further comprises suitable packaging to safely transport the buffer and other components.

The present invention also relates in part to an electroporation apparatus comprising: one or more chambers; one or more pairs of electrodes configured to generate electric fields within the one or more chambers, each electric field corresponding to one chamber; and a flow channel. In some embodiments, the apparatus further comprises: an inlet port; an outlet port; and a flanking flow channel connecting the inlet port and the outlet port to the flow channel.

In some embodiments, the electroporation apparatus further comprises: a pump for pumping a liquid medium from the flow channel into at least one of the one or more chambers during a collection process, wherein the liquid medium is obtained at the inlet port. In certain embodiments, the pump may further comprise one or more valves connecting the one or more chambers to the flow channel. In certain embodiments, the one or more valves may be capable of opening one at a time. In certain embodiments, the one or more valves permits only one-directional flow of fluid. In certain embodiments, each of the one or more valves corresponds to one chamber of the one or more of chambers. In certain embodiments, one or more valves is a pinch-valve or pinch-type valve. In certain embodiments, one or more valves operates using a spring motion, a lever motion, or a piston motion. In certain embodiments, each of the one or more chambers has a shape that narrows toward the one or more valves.

In some embodiments, the electroporation apparatus may also comprise one or more openings on its surface leading to the one or more chambers and an airflow channel below the one or more openings that connects the airflow between the one or more chambers. In certain embodiments, the electroporation apparatus further comprises a vent or air filter connecting the airflow channel to an exterior of the electroporation apparatus. In certain embodiments, the electroporation apparatus further comprises a seal configured to cover the one or more openings.

In some embodiments of the electroporation apparatus, each of the one or more chambers comprises a pair of electrodes comprising: a first electrode located on one side of a chamber; and a second electrode located on the opposite side of that chamber. In certain such embodiments, each electrode comprises an interior portion inside its corresponding chamber and an exterior portion external to its corresponding chamber. In certain embodiments, the interior portion may have an elliptical face and comprises a gold coating. In certain embodiments, each electrode pair is configured to connect to an electric circuit. In certain embodiments, each of the one or more chambers comprises a gap distance of about 0.1 mm to about 20 mm, about 0.5 mm to about 10 mm, about 1 mm to about 7 mm, or about 1 mm to about 4 mm. In certain embodiments, each chamber comprises a gap distance of less than about 4 mm.

In some embodiments of the electroporation apparatus, each of the one or more chambers may be configured to store a volume of at least about 50 μL, at least about 100 μL, at least about 150 at least about L, at least about 200 μL, at least about 250 μL, at least about 300 μL, at least about 350 μL, at least about 400 μL, at least about 450 μL, at least about 150 μL, at least about 500 μL, at least about 550 μL, at least about 600 μL, at least about 650 μL, at least about 700 μL, at least about 750 μL, at least about 800 μL, at least about 850 μL, at least about 900 μL, at least about 950 μL, or at least about 1000 μL (1.0 mL). In certain such embodiments, a chamber is configured to store a volume of at least about 250 μL or at least about 500 μL. In certain embodiments, the one or more chambers, in combination, are configured to store at least about 500 L, at least about 1.0 mL, at least about 1.2 mL, at least about 1.4 mL, at least about 1.6 mL, at least about 1.8 mL, at least about 2.0 mL, at least about 2.2 mL, at least about 2.4 mL, at least about 2.6 mL, at least about 2.8 mL, at least about 3.0 mL, at least about 3.2 mL, at least about 3.4 mL, at least about 3.6 mL, at least about 3.8 mL, at least about 4.0 mL, at least about 4.2 mL, at least about 4.4 mL, at least about 4.6 mL, at least about 4.8 mL, at least about 5.0 mL, at least about 5.2 mL, at least about 5.4 mL, at least about 5.6 mL, at least about 5.8 mL, at least about 6.0 mL, at least about 6.2 mL, at least about 6.4 mL, at least about 6.6 mL, at least about 6.8 mL, or at least about 7.0 mL of cells in liquid suspension for electroporation. In certain such embodiments, the one or more chambers, in combination, are configured to store at least 2 mL, at least 2.4 mL, at least 3.2 mL, at least 4 mL, at least 4.8 mL, at least 5.6 mL, or at least 6.4 mL of cells in liquid suspension for electroporation.

The present invention also relates in part to a method for electroporation using the apparatus of the present invention. In some embodiments, the method comprises using a pair of electrodes to generate an electric field in a chamber. In certain embodiments, the method further comprises opening one or more valves connected to the one or more chambers of the apparatus, thereby executing a cell collection process. In certain embodiments of the method, each valve is opened one at a time. In certain embodiments, the method further comprises transporting the buffer and cells to one or more outlet ports using one or more flow channels connected to the one or more valves. In certain embodiments, the method further comprises using one or more pumps to pump a liquid medium from a flow channel into at least one of the chambers, wherein the liquid medium is obtained at one or more inlet ports. In certain embodiments, the method further comprises draining two or more chambers into a flow channel. In certain embodiments, the method further comprises depositing cells into one or more openings of the electroporation apparatus leading to the one or more chambers containing the buffer, applying a seal to each opening, and connecting one or more pairs of electrodes to at least one circuit by inserting the electroporation apparatus into a docking station.

The present invention also relates in part to a method of electroporation using the buffer of the present invention and an UltraPorator™ device.

The present invention also relates in part to the use of a buffer of the present invention in the electroporation apparatus of the present invention. In certain embodiments, the use involves an electroporation method of the present invention.

The present invention also relates in part to a system for electroporation, the system comprising: the buffer of the present invention; and an apparatus of the present invention. In some embodiments, the buffer comprises: a buffer selected from Tables 2 and 3, for example Buffer 1, Buffer 2, or Buffer 3, as set forth in Table 2. In certain embodiments, the apparatus comprises an UltraPorator™ electroporation apparatus. In certain embodiments, the system is used to transfect cells, for example lymphocytes such as T cells. In certain embodiments, the system produces a higher cell transfection efficiency rate as compared to a system that comprises the same apparatus and a commercially available electroporation buffer.

### BRIEF DESCRIPTION OF THE DRAWINGS

The features of the present disclosure are set forth with particularity in the appended claims. The following description accompanies the drawings, all given by way of non-limiting examples that may be useful to understand how the described buffer and methods of use may be embodied.

FIG. 1 is a graph showing the yield percentage of three electroporation buffers, designated Buffers 1, 2, and 3, along with a control electroporation buffer, on cells collected from three donors, designated Donors 1, 2, and 3.

FIG. 2 is a graph showing the overall electroporation performance of the three sample electroporation buffers (Buffers 1, 2, and 3) and a control electroporation buffer on Donor 1's cells. Electroporation performance results are provided in terms of viability percentage, recovery percentage, transfection percentage, and yield percentage.

FIG. 3 is a graph showing the electroporation performance of the three sample electroporation buffers (Buffers 1, 2, and 3) and a control electroporation buffer on Donor 2's cells. Electroporation performance results are provided in terms of viability percentage, recovery percentage, transfection percentage, and yield percentage.

FIG. 4 is a graph showing the electroporation performance of the three sample electroporation buffers (Buffers 1, 2, and 3) and a control electroporation buffer on Donor 3's cells. Electroporation performance results are provided in terms of viability percentage, recovery percentage, transfection percentage, and yield percentage.

FIG. 5 is a graph showing the electroporation performance of the three sample electroporation buffers (Buffers 1, 2, and 3) and a control electroporation buffer on Donor 1 and Donor 2's cells. Electroporation performance results are provided in terms of yield percentage for the uptake of a CAR2 construct.

### DETAILED DESCRIPTION OF THE INVENTION

Minimal component electroporation buffers, methods of use, and kits are provided. The buffers are capable of facilitating the introduction of biological material dissolved or suspended therein into a population of cells via an electric current. As will be described in more detail, the disclosed buffer embodiments are capable of introducing biological materials into a population of cells with improved transfection efficiencies, cell viability, and/or cell yield as compared to control buffer. Further, it is unexpected that the disclosed minimal buffer components, would be sufficient for high transfection efficiency. The minimal components of the buffer save resources producing and controlling the quality of the electroporation buffers.

Before the present invention is described in greater detail, it is to be understood that this invention is not limited to particular cases described. It is also to be understood that the terminology used herein is for the purpose of describing particular embodiments only, and is not intended to be limiting, since the scope of the present invention will be limited only by the appended claims.

### Ranges and Definitions

Unless defined otherwise herein, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used herein, the singular forms “a”, “an”, and “the” include plural referents unless the context clearly dictates otherwise. Similarly, the phrase “one or more” means at least one and also includes plural referents, again unless the context clearly dictates otherwise.

As used herein, the terms “and/or” and “any combination thereof” and their grammatical equivalents may be used interchangeably. Solely for illustrative purposes, the following phrases “A, B, and/or C” or “A, B, C, or any combination thereof” can mean “A individually; B individually; C individually; A and B; B and C; A and C; and A, B, and C.”

As used herein, the term “about” in relation to a reference numerical value and its grammatical equivalents includes the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.” For example, the phrase “about 45 mM, 40 mM, 35 mM,” and so on means “about 45 mM, about 40 mM, about 35 mM,” and so on.

As used herein, the term “cell” refers to a prokaryotic or eukaryotic cell that can be, or has been, used as a recipient for a nucleic acid (e.g., an expression vector that comprises a nucleotide sequence encoding one or more gene products (for example chimeric antigen receptor (CAR) gene products), and includes any progeny of the original cell that has been genetically modified by the nucleic acid. A “recombinant cell” or “genetically modified cell” is a cell into which has been introduced an exogenous nucleic acid.

As used herein, the term “biological material” refers to material derived from a biological source. A “biological material” according to the invention may consist of isolated or purified nucleic acids (including RNA and DNA; whether single or double-stranded, also including hybrid and chimeric forms thereof), proteins, peptides, ribonucleoproteins (RNPs), or other naturally occurring polymers. The biological material may be animal, plant, bacterial, yeast, or viral material containing a particular nucleic acid of interest.

As used herein, the term “exogenous” and its grammatical equivalents means derived from a different source that the reference source. For example, an immune cell comprising an “exogenous” nucleic acid is an immune cell that comprises a nucleic acid from a source that is not the immune cell itself. The nucleic acid may be from a different immune cell, or from some other cell. The nucleic acid may even be from a different organism or even species, for example from a eukaryotic, bacterial, plant, or yeast source.

As used herein, the term “recombinant” and its grammatical equivalents means that a particular biological material (e.g., nucleic acid or peptide) is the product of various combinations of cloning, restriction, and/or ligation steps resulting in a construct distinguishable from endogenous biological materials found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

As used herein, the term “recombinant nucleic acid” refer to a nucleic acid that is non-naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

As used herein the term “isolated” refers to a compound, nucleic acid, polypeptide, or cell that is in an environment different from that in which the compound, nucleic acid, polypeptide, or cell naturally occurs.

As will be apparent to those of skill in the art upon reading this disclosure, each of the individual embodiments described and illustrated herein has discrete components and features, which can be readily separated from or combined with the features of any of the other several cases without departing from the scope or spirit of the present invention. Any recited method can be carried out in the order of events recited or in any other order that is logically possible.

### Electroporation (EP) Buffers

The buffers disclosed herein were found to have improved properties, including enhanced transfection capabilities, notwithstanding that these buffers comprise fewer components as compared to other known electroporation buffers.

In some embodiments, the buffer comprises a solvent, such as water. In some embodiments, the water may be purified and/or sterilized. For example, the water may be subjected to deionization (e.g., capacitive deionization or electrodeionization), reverse osmosis, carbon filtering, microfiltration, ultrafiltration, and/or ultraviolet sterilization. In some embodiments, the water is deionized. In some embodiments, the water is of a quality designated as “water for injection”; also known as “sterile water for injection.” Water for injection is generally made by distillation or reverse osmosis. Water for injection is a sterile, nonpyrogenic, solute-free preparation of water, chemically designated “H₂O,” and having a pH of between about 5.0 and about 7.0, preferably about 5.5.

In some embodiments, the solvent comprises between 0.1% and 99.9% by volume of the total buffer volume. For example, the solvent may comprise at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, or 99.1% by volume of the total buffer volume.

In some embodiments, the buffer comprises a solute, for example a sugar or an organic compound derived from sugar, for example a sugar alcohol. In embodiments wherein the buffer comprises a sugar, the sugar may comprise a monosaccharide, a disaccharide, and/or a polysaccharide. In some embodiments, the sugar comprises a monosaccharide, for example glucose, fructose, and/or galactose. In some embodiments, the sugar comprises a disaccharide, for example sucrose, lactose, and maltose. In some embodiments, the sugar comprises a polysaccharide, for example cellulose or starch. In embodiments wherein the buffer comprises a sugar alcohol, the sugar alcohol may comprise mannitol, sorbitol, xylitol, lactitol, isomalt, maltitol, and/or hydrogenated starch hydrolysates (HSH).

In some embodiments, the sugar is present in an amount less than about 50 millimolar (mM). For example, the sugar may be present in an amount less than about 45 mM, 40 mM, 35 mM, 30 mM, 25 mM, 20 mM, 15 mM, 10 mM, or 5 mM. In some embodiments, the sugar is present in an amount that ranges between about 10 mM to about 50 mM, about 10 mM to about 40 mM, about 10 mM to about 20 mM, about 20 mM to about 40 mM, about 25 mM to about 35 mM, about 26 mM to about 36 mM, about 26 mM to about 34 mM, about 27 mM to about 35 mM, about 27 mM to about 33 mM, about 28 mM to about 34 mM, about 28 mM to about 32 mM, about 29 mM to about 33 mM, about 29 mM to about 31 mM, about 30 mM to about 32 mM, about 29.1 mM to about 30.9 mM, about 30.1 mM to about 31.9 mM, about 29.2 mM to about 30.8 mM, about 30.2 mM to about 31.8 mM, about 29.3 mM to about 30.7 mM, about 30.3 mM to about 31.7 mM, about 29.4 mM to about 30.6 mM, about 30.4 mM to about 31.6 mM, about 29.5 mM to about 30.5 mM, about 30.5 mM to about 31.5 mM, about 29.6 mM to about 30.4 mM, about 30.6 mM to about 31.4 mM, about 29.7 mM to about 30.3 mM, about 30.7 mM to about 31.3 mM, about 29.8 mM to about 30.2 mM, about 30.8 mM to about 31.2 mM, about 29.9 mM to about 30.1 mM, or about 30.9 mM to about 31.1 mM. In some embodiments, the sugar is present in an amount of about 25 mM, 26 mM, 27 mM, 28 mM, 29 mM, 30 mM, 31 mM, 32 mM, 33 mM, 34 mM, or 35 mM.

In some embodiments, the sugar is glucose. In these embodiments, the glucose may be present in an amount less than about 50 millimolar (mM). For example, the glucose may be present in an amount less than about 45 mM, 40 mM, 35 mM, 30 mM, 25 mM, 20 mM, 15 mM, 10 mM, or 5 mM. In some embodiments, the glucose is present in an amount that ranges between about 10 mM and about 50 mM, about 10 mM and about 40 mM, about 10 mM and about 20 mM, or about 25 mM and about 35 mM. In certain embodiments, glucose is present in an amount of about 20 mM to about 40 mM, from about 25 mM to about 35 mM, about 26 mM to about 36 mM, about 26 mM to about 34 mM, about 27 mM to about 35 mM, about 27 mM to about 33 mM, about 28 mM to about 34 mM, about 28 mM to about 32 mM, about 29 mM to about 33 mM, about 29 mM to about 31 mM, about 30 mM to about 32 mM, about 29.1 mM to about 30.9 mM, about 30.1 mM to about 31.9 mM, about 29.2 mM to about 30.8 mM, about 30.2 mM to about 31.8 mM, about 29.3 mM to about 30.7 mM, about 30.3 mM to about 31.7 mM, about 29.4 mM to about 30.6 mM, about 30.4 mM to about 31.6 mM, about 29.5 mM to about 30.5 mM, about 30.5 mM to about 31.5 mM, about 29.6 mM to about 30.4 mM, about 30.6 mM to about 31.4 mM, about 29.7 mM to about 30.3 mM, about 30.7 mM to about 31.3 mM, about 29.8 mM to about 30.2 mM, about 30.8 mM to about 31.2 mM, about 29.9 mM to about 30.1 mM, or about 30.9 mM to about 31.1 mM. In some embodiments, the glucose is present in an amount of about 25 mM, 26 mM, 27 mM, 28 mM, 29 mM, 30 mM, 31 mM, 32 mM, 33 mM, 34 mM, or 35 mM. In certain embodiments, the glucose is present in an amount of about 30 mM or 31 mM.

In some embodiments, the sugar is mannitol. In these embodiments, the mannitol may be present in an amount less than about 50 millimolar (mM). For example, the mannitol may be present in an amount less than about 45 mM, 40 mM, 35 mM, 30 mM, 25 mM, 20 mM, 15 mM, 10 mM, or 5 mM. In some embodiments, the mannitol is present in an amount that ranges between about 10 mM and about 50 mM, about 10 mM and about 40 mM, about 10 mM and about 20 mM, or about 25 mM and about 35 mM. In certain embodiments, mannitol is present in an amount of about 20 mM to about 40 mM, about 25 mM to about 35 mM, about 26 mM to about 36 mM, about 26 mM to about 34 mM, about 27 mM to about 35 mM, about 27 mM to about 33 mM, about 28 mM to about 34 mM, about 28 mM to about 32 mM, about 29 mM to about 33 mM, about 29 mM to about 31 mM, about 30 mM to about 32 mM, about 29.1 mM to about 30.9 mM, about 30.1 mM to about 31.9 mM, about 29.2 mM to about 30.8 mM, about 30.2 mM to about 31.8 mM, about 29.3 mM to about 30.7 mM, about 30.3 mM to about 31.7 mM, about 29.4 mM to about 30.6 mM, about 30.4 mM to about 31.6 mM, about 29.5 mM to about 30.5 mM, about 30.5 mM to about 31.5 mM, about 29.6 mM to about 30.4 mM, about 30.6 mM to about 31.4 mM, about 29.7 mM to about 30.3 mM, about 30.7 mM to about 31.3 mM, about 29.8 mM to about 30.2 mM, about 30.8 mM to about 31.2 mM, about 29.9 mM to about 30.1 mM, or about 30.9 mM to about 31.1 mM. In some embodiments, the mannitol is present in an amount of about 25 mM, 26 mM, 27 mM, 28 mM, 29 mM, 30 mM, 31 mM, 32 mM, 33 mM, 34 mM, or 35 mM. In certain embodiments, the glucose is present in an amount of about 30 mM or 31 mM.

In some embodiments, the EP buffer comprises one or more chloride salts, for example potassium chloride (KCl) and/or magnesium chloride (MgCl₂).

In some embodiments, the buffer further comprises one or more buffering agents, for example, dibasic sodium phosphate (Na₂HPO₄), monobasic sodium phosphate(NaH₂PO₄), or a combination of Na₂HPO₄ and NaHPO₄ (referred to interchangeably as Na₂HPO₄/NaH₂PO₄ or sodium phosphate). In some embodiments, the buffer further comprises one or more of 4-(2-hydroxyethyl)-1-piperazineethanesulfonic acid (HEPES), tris(hydroxymethyl)aminomethane or tris(hydroxymethyl)methylamine (“Tris”), and/or dimethyl sulfoxide (DMSO). In other embodiments, the buffer specifically excludes one or more buffering agents commonly found in commercial electroporation (EP) buffers. For example, in some embodiments, the buffer excludes one or more of DMSO, Tris, and/or HEPES.

In some embodiments, the buffer comprises a solvent, one or more chloride salts, and one or more buffering agents. In some such embodiments: the solvent is water; the sugar is glucose or mannitol; the one or more chloride salts comprises KCl and/or MgCl₂; and/or the one or more buffering agents comprises HEPES, DMSO, and/or sodium phosphate. In certain embodiments, the buffer does not comprise DMSO and/or HEPES. In some embodiments, the buffer comprises, consists essentially of, or consists of: water; glucose and/or mannitol; KCl and/or MgCl₂; and sodium phosphate. In certain embodiments, the buffer comprises, consists essentially of, or consists of: water; glucose and/or mannitol; KCl and/or MgCl₂; sodium phosphate; and HEPES and/or DMSO.

In some embodiments, the buffer comprises water (H₂O), glucose, KCl, MgCl₂, and Na₂HPO₄/NaH₂PO₄. In some embodiments, the buffer comprises water (H₂O), glucose, KCl, MgCl₂, Na₂HPO₄/NaH₂PO₄, and HEPES. In other embodiments, the buffer comprises water (H₂O), glucose, KCl, MgCl₂, Na₂HPO₄/NaH₂PO₄, HEPES, and DMSO.

In some embodiments, the buffer consists essentially of water (H₂O), glucose, KCl, MgCl₂, and Na₂HPO₄/NaH₂PO₄. In some embodiments, the buffer consists essentially of water (H₂O), glucose, KCl, MgCl₂, Na₂HPO₄/NaH₂PO₄, and HEPES. In other embodiments, the buffer consists essentially of water (H₂O), glucose, KCl, MgCl₂, Na₂HPO₄/NaH₂PO₄, HEPES, and DMSO.

In some embodiments, the buffer consists of water (H₂O), glucose, KCl, MgCl₂, and Na₂HPO₄/NaH₂PO₄. In some embodiments, the buffer consists of water (H₂O), glucose, KCl, MgCl₂, Na₂HPO₄/NaH₂PO₄, and HEPES. In other embodiments, the buffer consists of water (H₂O), glucose, KCl, MgCl₂, Na₂HPO₄/NaH₂PO₄, HEPES, and DMSO.

In some embodiments, the buffering agent has a pH ranging from about 6.0 to 8.0, 6.5 to 8.0, 7.0 to 8.0, 7.5 to 8.0, 6.0 to 7.5, 6.0 to 7.0, 6.0 to 6.5, 6.5 to 7.5, or 6.5 to 7.0. In some embodiments, the buffering agent has a pH of from about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the buffering agent has a pH of about 6.5, 6.6, 6.7, 6.8, 6.9, 7.0, 7.1, 7.2, 7.3, 7.4, 7.5, 7.6, 7.7., 7.8, 7.9, or 8.0.

In some embodiments, the buffer comprising the one or more buffering agents has a pH ranging from about 6.0 to 8.0, 6.5 to 8.0, 7.0 to 8.0, 7.5 to 8.0, 6.0 to 7.5, 6.0 to 7.0, 6.0 to 6.5, 6.5 to 7.5, or 6.5 to 7.0. In some embodiments, the buffer has a pH of about 6.5, 6.6., 6.7, 6.8, 6.9, 7.0, 7.1, 7.2, 7.3, 7.4, 7.5, 7.6, 7.7., 7.8, 7.9, or 8.0. In some embodiments, the buffer has a pH of from about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2.

In some embodiments, the buffer comprises one or both of Na₂HPO₄ and/or NaH₂PO₄. In embodiments wherein the buffer comprises both buffering agents, the ratio of the two (i.e., Na₂HPO₄/NaH₂PO₄) may be about 1:1, 1:2, 1:3, 1:4, 1:5, 1:6, 1:7, 1:8, 1:9, 9:1, 8:1, 7:1, 6:1 5:1, 4:1, 3:1, 2:1, or 2:3.

In some embodiments, the Na₂HPO₄/NaH₂PO₄ has a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the Na₂HPO₄/NaH₂PO₄ has a pH of 6.5, 6.6., 6.7, 6.8, 6.9, 7.0, 7.1, 7.2, 7.3, 7.4, 7.5, 7.6, 7.7, 7.8, or 7.9.

In some embodiments, a mixture of Na₂HPO₄ and NaH₂PO₄ (also referred to “Na₂HPO₄/NaH₂PO₄” or “sodium phosphate”) may be present in the buffer in an amount ranging from about 50 mM and 160 mM, 60 mM to 150 mM, 70 mM to 140 mM, 75 mM to 130 mM, 80 mM to 125 mM, 90 mM to 125 mM, 90 mM to 120 mM, 90 mM to 115 mM, or 90 mM to 105 mM. In certain embodiments, Na₂HPO₄/NaH₂PO₄ is present in an amount of about 90 mM to about 120 mM. In certain embodiments, Na₂HPO₄/NaH₂PO₄ is present in an amount of about 95 mM to about 115 mM, about 100 mM to about 110 mM, about 101 mM to about 109 mM, about 102 mM to about 108 mM, about 103 mM to about 107 mM, about 104 mM to about 106 mM, about 104.1 mM to about 105.9 mM, about 104.2 mM to about 105.8 mM, about 104.3 mM to about 105.7 mM, about 104.4 mM to about 105.6 mM, about 104.5 mM to about 105.5 mM, about 104.6 mM to about 105.4 mM, about 104.7 mM to about 105.3 mM, about 104.8 mM to about 105.2 mM, or about 104.9 mM to about 105.1 mM. In certain embodiments, Na₂HPO₄/NaH₂PO₄ is present in an amount of about 80 mM to about 100 mM, about 85 mM to about 95 mM, about 86 mM to about 94 mM, about 87 mM to about 93 mM, about 88 mM to about 92 mM, about 89 mM to about 91 mM, about 89.1 mM to about 90.9 mM, about 89.2 mM to about 90.8 mM, about 89.3 mM to about 90.7 mM, about 89.4 mM to about 90.6 mM, about 89.5 mM to about 90.5, about 89.6 mM to about 90.4 mM, about 89.7 mM to about 90.3 mM, about 89.8 mM to about 90.2 mM, or about 89.9 mM to about 90.1 mM. In certain embodiments, Na₂HPO₄/NaH₂PO₄ is present in an amount of about 90 mM or about 105 mM. In some embodiments, the Na₂HPO₄/NaH₂PO₄ is present in an amount of at least 50 mM, 60 mM, 70 mM, 80 mM, 90 mM, or 100 mM. In some embodiments, the Na₂HPO₄/NaH₂PO₄ is present in an amount of 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, or 120 mM. In certain embodiments, the Na₂HPO₄/NaH₂PO₄ is present in an amount of 90 mM or 105 mM.

In embodiments wherein the buffer comprises KCl, KCl may be present in an amount less than about 30 mM. For example, KCl may be present in an amount less than about 25 mM, 20 mM, 15 mM, 10 mM, or 5 mM. In some embodiments, KCl is present in an amount that ranges between about 1 mM and about 30 mM, about 2 mM and about 25 mM, about 3 mM and about 20 mM, about 4 mM and about 15 mM, about 5 mM and about 10 mM, or about 5 mM to about 15 mM. In some embodiments, KCl is present in an amount of 0 to about 15 mM, 0 to about 10 mM, about 1 mM to about 9 mM, about 2 mM to about 8 mM, about 3 mM to about 7 mM, about 4 mM to about 6 mM, about 4.1 mM to about 5.9 mM, about 4.2 mM to about 5.8 mM, about 4.3 mM to about 5.7 mM, about 4.4 mM to about 5.6 mM, about 4.5 mM to about 5.5 mM, about 4.6 mM to about 5.4 mM, about 4.7 mM to about 5.3 mM, about 4.8 mM to about 5.2 mM, or about 4.9 mM to about 5.1 mM. In some embodiments, KCl is present in an amount of 0 to about 20 mM, about 5 mM to about 15 mM, about 6 mM to about 14 mM, about 7 mM to about 13 mM, about 8 mM to about 12 mM, about 9 mM to about 11 mM, about 9.1 mM to about 10.9 mM, about 9.2 mM to about 10.8 mM, about 9.3 mM to about 10.7 mM, about 9.4 mM to about 10.6 mM, about 9.5 mM to about 10.5 mM, about 9.6 mM to about 10.4 mM, about 9.7 mM to about 10.3 mM, about 9.8 mM to about 10.2 mM, or about 9.9 mM to about 10.1 mM. In some embodiments, the KCl is present in an amount of about 1 mM, 2 mM, 3 mM, 4 mM, 5 mM, 6 mM, 7 mM, 8 mM, 9 mM, 10 mM, 11 mM, 12 mM, 13 mM, 14 mM, or 15 mM. In certain embodiments, KCl is present in an amount of from about 5 mM to about 15 mM. In certain embodiments, the KCl is present in an amount of about 5 mM or 10 mM.

In some embodiments, the KCl has a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the KCl has a pH of about 6.5, 6.6, 6.7, 6.8, 6.9, 7.0, 7.1, 7.2, 7.3, 7.4, 7.5, 7.6, 7.7, 7.8, or 7.9.

In embodiments wherein the buffer comprises MgCl₂, MgCl₂ may be present in an amount less than about 50 mM. For example, MgCl₂ may be present in an amount less than about 45 mM, 35 mM, 30 mM 25 mM, 20 mM, 15 mM, 10 mM, or 5 mM. In some embodiments, MgCl₂ is present in an amount that ranges between about 5 mM and about 50 mM, about 6 mM and about 45 mM, about 7 mM and about 40 mM, about 8 mM and about 35 mM, about 9 mM and about 30 mM, about 10 mM and about 25 mM, or about 15 mM and about 25 mM. In some embodiments, MgCl₂ is present in an amount of from about 5 mM to about 25 mM, about 10 mM to about 20 mM, about 11 mM to about 19 mM, about 12 mM to about 18 mM, about 13 mM to about 17 mM, about 14 mM to about 16 mM, about 14.1 mM to about 15.9 mM, about 14.2 mM to about 15.8 mM, about 14.3 mM to about 15.7 mM, about 14.4 mM to about 15.6 mM, about 14.5 mM to about 15.5 mM, about 14.6 mM to about 15.4 mM, 14.7 mM to about 15.3 mM, 14.8 mM to about 15.2 mM, or about 14.9 mM to about 15.1 mM. In some embodiments, MgCl₂ is present in an amount of from about 10 mM to about 30 mM, about 15 mM to about 25 mM, about 16 mM to about 24 mM, about 17 mM to about 23 mM, about 18 mM to about 22 mM, about 19 mM to about 21 mM, about 19.1 mM to about 20.9 mM, about 19.2 mM to about 20.8 mM, about 19.3 mM to about 20.7 mM, about 19.4 mM to about 20.6 mM, about 19.5 mM to about 20.5 mM, about 19.6 mM to about 20.4 mM, about 19.7 mM to about 20.3 mM, about 19.8 mM to about 20.2 mM, or about 19.9 mM to about 20.1 mM. In some embodiments, the MgCl₂ is present in an amount of about 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, or 30 mM. In certain embodiments, the MgCl₂ is present in an amount of about 15 mM or 20 mM. In certain embodiments, MgCl₂ is present in an amount of about 10 mM or about 15 mM.

In some embodiments, the MgCl₂ has a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the MgCl₂ has a pH of about 6.5, 6.6., 6.7, 6.8, 6.9, 7.0, 7.1, 7.2, 7.3, 7.4, 7.5, 7.6, 7.7, 7.8, or 7.9.

In embodiments wherein the buffer comprises HEPES, HEPES may be present in an amount less than about 30 mM. For example, HEPES may be present in an amount less than about 25 mM, 20 mM, 15 mM, 10 mM, 5 mM, 4 mM, 3 mM, 2 mM, 1 mM, 0.5 mM, or 0.1 mM. In some embodiments, HEPES is present in an amount that ranges between about 1 mM and about 30 mM, about 2 mM and about 25 mM, about 3 mM and about 20 mM, about 4 mM and about 15 mM, about 5 mM and about 10 mM. In some embodiments, HEPES is present in an amount of 0 to about 15 mM, 0 to about 10 mM, about 1 mM to about 9 mM, about 2 mM to about 8 mM, about 3 mM to about 7 mM, about 4 mM to about 6 mM, about 4.1 mM to about 5.9 mM, about 4.2 mM to about 5.8 mM, about 4.3 mM to about 5.7 mM, about 4.4 mM to about 5.6 mM, about 4.5 mM to about 5.5 mM, about 4.6 mM to about 5.4 mM, about 4.7 mM to about 5.3 mM, about 4.8 mM to about 5.2 mM, or about 4.9 mM to about 5.1 mM. In some embodiments, HEPES is present in an amount of 0 to about 20 mM, about 5 mM to about 15 mM, about 6 mM to about 14 mM, about 7 mM to about 13 mM, about 8 mM to about 12 mM, about 9 mM to about 11 mM, about 9.1 mM to about 10.9 mM, about 9.2 mM to about 10.8 mM, about 9.3 mM to about 10.7 mM, about 9.4 mM to about 10.6 mM, about 9.5 mM to about 10.5 mM, 9.6 mM to about 10.4 mM, about 9.7 mM to about 10.3 mM, about 9.8 mM to about 10.2 mM, or about 9.9 mM to about 10.1 mM. In certain embodiments, HEPES is present in an amount of about 5 mM to about 10 mM. In some embodiments, the HEPES is present in an amount of about 0.1 mM, 0.5 mM, 1 mM, 2 mM, 3 mM, 4 mM, 5 mM, 6 mM, 7 mM, 8 mM, 9 mM, 10 mM, 11 mM, 12 mM, 13 mM, 14 mM, or 15 mM. In certain embodiments, the HEPES is present in an amount of 0 mM, about 5 mM, or about 10 mM.

In some embodiments, the HEPES has a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the HEPES has a pH of about 6.5, 6.6, 6.7, 6.8, 6.9, 7.0, 7.1, 7.2, 7.3, 7.4, 7.5, 7.6, 7.7, 7.8, or 7.9.

In embodiments wherein the buffer comprises DMSO, the DMSO may be present in an amount equal to or less than 5%, 4%, 3%, 2%, 1%, 0.9%, 0.8%, 0.7%, 0.6%, 0.5%, 0.4%, 0.3%, 0.2%, or 0.1% by volume of the total buffer volume. In some embodiments, DMSO is present from about 0% to about 2.5% by volume of the total buffer volume. In some embodiments, DMSO is present in an amount ranging from about 0.1% to 5%, 1% to 5%, 2% to 5%, 3% to 5%, or 4% to 5% by volume of the total buffer volume.

In some embodiments, the DMSO has a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the DMSO has a pH of about 6.5, 6.6, 6.7, 6.8, 6.9, 7.0, 7.1, 7.2, 7.3, 7.4, 7.5, 7.6, 7.7, 7.8, or 7.9. In other embodiments, DMSO is not included in the buffer at all.

In some embodiments the buffer may comprise Tris in addition to, or in lieu of, one or more other components, such as HEPES. In certain embodiments, the Tris is present in an amount ranging from about 1 mM to 1M, 10 mM to 500 mM, 25 mM to 250 mM, or 50 mM to 100 mM. In certain embodiments, the Tris is present in an amount of about 1 mM, 5 mM, 10 mM, 15 mM 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In certain embodiments, the pH of the Tris in the buffer may be adjusted by adding one or more salts, such as HCl. In some embodiments, the Tris has a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the Tris has a pH of about 6.5, 6.6, 6.7, 6.8, 6.9, 7.0, 7.1, 7.2, 7.3, 7.4, 7.5, 7.6, 7.7, 7.8, or 7.9. In other embodiments, DMSO is not included in the buffer at all.

In certain embodiments, the buffer comprises a sugar in an amount equal to or less than 50 mM; HEPES in an amount equal to or less than 25 mM; Na₂HPO₄/NaH₂PO₄ in an amount equal to or less than 160 mM; KCl in an amount equal to or less than 10 mM; MgCl₂ in an amount equal to or less than 20 mM; and DMSO in an amount equal to or less than 5% by volume of the total buffer volume. In some of these embodiments, the sugar may comprise a monosaccharide and/or a sugar alcohol. In some of these embodiments, the sugar is mannitol and/or glucose. In some of these embodiments, the sugar is glucose. In some embodiments, the buffer does not comprise DMSO.

In certain embodiments, the buffer comprises a sugar in an amount of at least about 15 mM; HEPES in an amount equal to or less than 25 mM; Na₂HPO₄/NaH₂PO₄ in an amount of at least about 90 mM; KCl in an amount of at least about 2 mM; MgCl₂ in an amount of at least 15 mM; and DMSO in an amount equal to or less than 5% by volume of the total buffer volume. In some of these embodiments, the sugar may comprise a monosaccharide and/or a sugar alcohol. In some of these embodiments, the sugar is mannitol and/or glucose. In some of these embodiments, the sugar is glucose. In some embodiments, the buffer does not comprise DMSO.

In certain embodiments, the buffer comprises a sugar in an amount ranging from about 15 mM to about 35 mM; KCl in an amount ranging from about 5 mM to about 10 mM; MgCl₂ in an amount ranging from about 10.5 mM to about 20 mM; Na₂HPO₄/NaH₂PO₄ in an amount ranging from about 90 mM to about 105 mM; HEPES in an amount equal to or less than 25 mM; and DMSO in an amount equal to or less than 5% by volume of the total buffer volume. In some of these embodiments, the sugar may comprise a monosaccharide and/or a sugar alcohol. In some of these embodiments, the sugar is mannitol and/or glucose. In some of these embodiments, the sugar is glucose. In some embodiments, the buffer does not comprise DMSO.

In certain embodiments, the buffer comprises glucose in an amount of about 15 mM; KCl in an amount of about 6 mM; MgCl₂ in an amount of about 10.5 mM Na₂HPO₄/NaH₂PO₄ in an amount of about 105 mM; HEPES in an amount ranging from about 15 mM; and DMSO in an amount of about 2.5% by volume of total buffer volume.

In some embodiments, the buffer comprises, consists essentially of, or consists of: water; glucose and/or mannitol in an amount of from about 25 mM to about 35 mM; KCl in an amount of from about 5 mM to about 15 mM; MgCl₂ in an amount of from about 15 mM to about 25 mM; and sodium phosphate in an amount of from about 90 mM to about 120 mM. In certain embodiments, the buffer comprises, consists essentially of, or consists of: water; glucose and/or mannitol in an amount of from about 25 mM to about 35 mM; KCl in an amount of from about 5 mM to about 15 mM; MgCl₂ in an amount of from about 15 mM to about 25 mM; sodium phosphate in an amount of from about 90 mM to about 120 mM; and HEPES in an amount of 0 mM to about 10 mM or from about 5 mM to about 10 mM and/or DMSO in an amount equal to or less than about 2.5% by volume of the total volume of the buffer.

In certain embodiments, the buffer comprises, consists essentially of, or consists of: water; glucose and/or mannitol in an amount of about 25 mM; KCl in an amount of about 15 mM; and MgCl₂ in an amount of about 25 mM; Na₂HPO₄/NaH₂PO₄ in an amount of about 120 mM; and HEPES in an amount of about 10 mM. In some embodiments, DMSO is specifically excluded from the buffer. In certain embodiments, the buffer comprises, consists essentially of, or consists of: water; glucose and/or mannitol in an amount of about 25 mM; KCl in an amount of about 15 mM; and MgCl₂ in an amount of about 25 mM; Na₂HPO₄/NaH₂PO₄ in an amount of about 120 mM; HEPES in an amount of about 10 mM; and DMSO in an amount equal to or less than about 2.5% by volume of the total volume of the buffer.

In certain embodiments, the pH of the buffer may be adjusted. In some embodiments, the buffer is adjusted to a pH of between 6.5 and 8. In some embodiments, the buffer is adjusted to a pH between about 7.0 and 7.6. In some embodiments, the buffer is adjusted to a pH of about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, about 6.6 to about 7.6, about 6.7 to about 7.5, about 6.8 to about 7.4, about 6.9 to about 7.3, or about 7.0 to about 7.2. In some embodiments, the buffer is adjusted to a pH between about 6.9 and 7.2, or between about 7.0 and 7.1. In some embodiments, the buffer has a pH of about 7.0 to about 7.1. In some embodiments, the buffer is adjusted to a pH of about 6.5, 6.6, 6.7, 6.8, 6.9, 7.0, 7.1, 7.2, 7.3, 7.4, 7.5, 7.6, 7.7, 7.8, 7.9, or 8.0. In certain embodiments, the buffer is adjusted to a pH of about 7.0 or 7.1.

In certain embodiments, the conductivity of the buffer is between about 7.0 millisiemens per centimeter (ms/cm) to about 16.0 ms/cm, about 9.0 ms/cm to about 16.0 ms/cm, about 11.0 ms/cm to about 16.0 ms/cm, or about 13.0 ms/cm to about 16.0 ms/cm. In some embodiments, the conductivity of the buffer is between about 7.0 ms/cm to about 15.0 ms/cm, about 9.0 ms/cm to about 15.0 ms/cm, about 11.0 ms/cm to about 15.0 ms/cm, or about 13.0 ms/cm to about 15.0 ms/cm. In some embodiments, the buffer has a conductivity of about 10.0 ms/cm to about 15.0 ms/cm.

In some embodiments, the conductivity of the buffer is about 13.3 ms/cm to about 15.3 ms/cm, about 13.4 ms/cm to about 15.2 ms/cm, about 13.5 ms/cm to about 15.1 ms/cm, about 13.6 ms/cm to about 15.0 ms/cm, about 13.7 ms/cm to about 14.9 ms/cm, about 13.8 ms/cm to about 14.8 ms/cm, about 13.9 ms/cm to about 14.7 ms/cm, about 14.0 ms/cm to about 14.6 ms/cm, about 14.1 ms/cm to about 14.5 ms/cm, or about 14.2 ms/cm to about 14.4 ms/cm. In some embodiments, the conductivity of the buffer is about 10.6 ms/cm to about 12.6 ms/cm, about 10.7 ms/cm to about 12.5 ms/cm, about 10.8 ms/cm to about 12.4 ms/cm, about 10.9 ms/cm to about 12.3 ms/cm, about 11.0 ms/cm to about 12.2 ms/cm, about 11.1 ms/cm to about 12.1 ms/cm, about 11.2 ms/cm to about 12.0 ms/cm, about 11.3 ms/cm to about 11.9 ms/cm, about 11.4 ms/cm to about 11.8 ms/cm, or about 11.5 ms/cm to about 11.7 ms/cm. In some embodiments, the conductivity of the buffer is about 11.8 ms/cm to about 13.8 ms/cm, about 11.9 ms/cm to about 13.7 ms/cm, about 12.0 ms/cm to about 13.6 ms/cm, about 12.1 ms/cm to about 13.5 ms/cm, about 12.2 ms/cm to about 13.4 ms/cm, about 12.3 ms/cm to about 13.3 ms/cm, about 12.4 ms/cm to about 13.2 ms/cm, about 12.5 ms/cm to about 13.1 ms/cm, about 12.6 ms/cm to about 13.0 ms/cm, or about 12.7 ms/cm to about 12.9 ms/cm. In some embodiments, the conductivity of the buffer is about 7.0 ms/cm, about 7.1 ms/cm, about 7.2 ms/cm, about 7.3 ms/cm, about 7.4 ms/cm, about 7.5 ms/cm, about 7.6 ms/cm, about 7.7 ms/cm, about 7.8 ms/cm, about 7.9 ms/cm, about 8.0 ms/cm, about 8.1 ms/cm, about 8.2 ms/cm, about 8.3 ms/cm, about 8.4 ms/cm, about 8.5 ms/cm, about 8.6 ms/cm, about 8.7 ms/cm, about 8.8 ms/cm, about 8.9 ms/cm, about 9.0 ms/cm, about 9.1 ms/cm, about 9.2 ms/cm, about 9.3 ms/cm, about 9.4 ms/cm, about 9.5 ms/cm, about 9.6 ms/cm, about 9.7 ms/cm, about 9.8 ms/cm, about 9.9 ms/cm, about 10.0 ms/cm, about 10.1 ms/cm, about 10.2 ms/cm, about 10.3 ms/cm, about 10.4 ms/cm, about 10.5 ms/cm, about 10.6 ms/cm, about 10.7 ms/cm, about 10.8 ms/cm, about 10.9 ms/cm, about 11.0 ms/cm, about 11.1 ms/cm, about 11.2 ms/cm, about 11.3 ms/cm, about 11.4 ms/cm, about 11.5 ms/cm, about 11.6 ms/cm, about 11.7 ms/cm, about 11.8 ms/cm, about 11.9 ms/cm, about 12.0 ms/cm, about 12.1 ms/cm, about 12.2 ms/cm, about 12.3 ms/cm, about 12.4 ms/cm, about 12.5 ms/cm, about 12.6 ms/cm, about 12.7 ms/cm, about 12.8 ms/cm, about 12.9 ms/cm, about 13.0 ms/cm, about 13.1 ms/cm, about 13.2 ms/cm, about 13.3 ms/cm, about 13.4 ms/cm, about 13.5 ms/cm, about 13.6 ms/cm, about 13.7 ms/cm, about 13.8 ms/cm, about 13.9 ms/cm, about 14.0 ms/cm, about 14.1 ms/cm, about 14.2 ms/cm, about 14.3 ms/cm, about 14.4 ms/cm, about 14.5 ms/cm, about 14.6 ms/cm, about 14.7 ms/cm, about 14.8 ms/cm, about 14.9 ms/cm, about 15.0 ms/cm, about 15.1 ms/cm, about 15.2 ms/cm, about 15.3 ms/cm, about 15.4 ms/cm, about 15.5 ms/cm, about 15.6 ms/cm, about 15.7 ms/cm, about 15.8 ms/cm, about 15.9 ms/cm, or about 16.0 ms/cm. In certain embodiments, the conductivity of the buffer is about 11.6, 12.8, or 14.3.

In some embodiments, the osmolality of the buffer is lower than the osmolality of the cells being transfected (i.e., also known as “intracellular osmolality”). In some embodiments, the osmolality of the buffer ranges from about 250 milliosmole per kilogram (mOsm/kg) H₂O to about 1255 mOsm/kg H₂O, about 250 mOsm/kg H₂O to about 1100 mOsm/kg H₂O, about 250 mOsm/kg H₂O to about 900 mOsm/kg H₂O, about 250 mOsm/kg H₂O to about 700 mOsm/kg H₂O, about 250 mOsm/kg H₂O to about 500 mOsm/kg H₂O, about 250 mOsm/kg H₂O to about 400 mOsm/kg H₂O, or about 250 mOsm/kg H₂O to about 360 mOsm/kg H₂O. In some embodiments, the osmolality is about 360 mOsm/kg H₂O to about 1255 mOsm/kg H₂O, about 360 mOsm/kg H₂O to about 1100 mOsm/kg H₂O, about 360 mOsm/kg H₂O to about 900 mOsm/kg H₂O, about 360 mOsm/kg H₂O to about 700 mOsm/kg H₂O, about 360 mOsm/kg H₂O to about 500 mOsm/kg H₂O, about 360 mOsm/kg H₂O to about 400 mOsm/kg H₂O. In certain such embodiments, the osmolality may be from about 275 mOsm/kgH₂O to about 350 mOsm/kgH₂O.

In some embodiments, the osmolality is from about 330 mOsm/kg H₂O to about 350 mOsm/kg H₂O, about 335 mOsm/kg H₂O to about 345 mOsm/kg H₂O, about 336 mOsm/kg H₂O to about 344 mOsm/kg H₂O, about 337 mOsm/kg H₂O to about 343 mOsm/kg H₂O, about 338 mOsm/kg H₂O to about 342 mOsm/kg H₂O, about 339 mOsm/kg H₂O to about 341 mOsm/kg H₂O, about 339.1 mOsm/kg H₂O to about 340.9 mOsm/kg H₂O, about 339.2 mOsm/kg H₂O to about 340.8 mOsm/kg H₂O, about 339.3 mOsm/kg H₂O to about 340.7 mOsm/kg H₂O, about 339.4 mOsm/kg H₂O to about 340.6 mOsm/kg H₂O, about 339.5 mOsm/kg H₂O to about 340.5 mOsm/kg H₂O, about 339.6 mOsm/kg H₂O to about 340.4 mOsm/kg H₂O, about 339.7 mOsm/kg H₂O to about 340.3 mOsm/kg H₂O, about 339.8 mOsm/kg H₂O to about 340.2 mOsm/kg H₂O, or about 339.9 mOsm/kg H₂O to about 340.1 mOsm/kg H₂O. In some embodiments, the osmolality is from about 270 mOsm/kg H₂O to about 290 mOsm/kg H₂O, about 275 mOsm/kg H₂O to about 285 mOsm/kg H₂O, about 276 mOsm/kg H₂O to about 284 mOsm/kg H₂O, about 277 mOsm/kg H₂O to about 283 mOsm/kg H₂O, about 278 mOsm/kg H₂O to about 282 mOsm/kg H₂O, about 279 mOsm/kg H₂O to about 281 mOsm/kg H₂O, about 279.1 mOsm/kg H₂O to about 280.9 mOsm/kg H₂O, about 279.2 mOsm/kg H₂O to about 280.8 mOsm/kg H₂O, about 279.3 mOsm/kg H₂O to about 280.7 mOsm/kg H₂O, about 279.4 mOsm/kg H₂O to about 280.6 mOsm/kg H₂O, about 279.5 mOsm/kg H₂O to about 280.5 mOsm/kg H₂O, about 279.6 mOsm/kg H₂O to about 280.4 mOsm/kg H₂O, about 279.7 mOsm/kg H₂O to about 280.3 mOsm/kg H₂O, about 279.8 mOsm/kg H₂O to about 280.2 mOsm/kg H₂O, or about 279.9 mOsm/kg H₂O to about 280.1 mOsm/kg H₂O. In some embodiments, the osmolality is from about 282 mOsm/kg H₂O to about 302 mOsm/kg H₂O, about 287 mOsm/kg H₂O to about 297 mOsm/kg H₂O, about 288 mOsm/kg H₂O to about 296 mOsm/kg H₂O, about 289 mOsm/kg H₂O to about 295 mOsm/kg H₂O, about 290 mOsm/kg H₂O to about 294 mOsm/kg H₂O, about 291 mOsm/kg H₂O to about 293 mOsm/kg H₂O, about 291.1 mOsm/kg H₂O to about 292.9 mOsm/kg H₂O, about 291.2 mOsm/kg H₂O to about 292.8 mOsm/kg H₂O, about 291.3 mOsm/kg H₂O to about 292.7 mOsm/kg H₂O, about 291.4 mOsm/kg H₂O to about 292.6 mOsm/kg H₂O, about 291.5 mOsm/kg H₂O to about 292.5 mOsm/kg H₂O, about 291.6 mOsm/kg H₂O to about 292.4 mOsm/kg H₂O, about 291.7 mOsm/kg H₂O to about 292.3 mOsm/kg H₂O, about 291.8 mOsm/kg H₂O to about 292.2 mOsm/kg H₂O, or about 291.9 mOsm/kg H₂O to about 292.1 mOsm/kg H₂O.

In some embodiments, the osmolality is about 250 mOsm/kg H₂O, 255 mOsm/kg H₂O, 260 mOsm/kg H₂O, 270 mOsm/kg H₂O, 275 mOsm/kg H₂O, about 280 mOsm/kg H₂O, about 285 mOsm/kg H₂O, about 290 mOsm/kg H₂O, about 300 mOsm/kg H₂O, about 305 mOsm/kg H₂O, about 310 mOsm/kg H₂O, about 315 mOsm/kg H₂O, about 320 mOsm/kg H₂O, about 325 mOsm/kg H₂O, about 330 mOsm/kg H₂O, about 335 mOsm/kg H₂O, about 340 mOsm/kg H₂O, about 345 mOsm/kg H₂O, about 350 mOsm/kg H₂O, about 355 mOsm/kg H₂O, about 360 mOsm/kg H₂O, about 365 mOsm/kg H₂O, about 370 mOsm/kg H₂O, about 375 mOsm/kg H₂O, about 380 mOsm/kg H₂O, about 385 mOsm/kg H₂O, about 390 mOsm/kg H₂O, about 395 mOsm/kg H₂O, or about 400 mOsm/kg H₂O. In certain embodiments, the osmolality is about 280 mOsm/kg H₂O, about 292 mOsm/kg H₂O, about 340 mOsm/kg H₂O, or about 362 mOsm/kg H₂O.
In some embodiments, the buffer comprises, consists essentially of, or consists of: water;
glucose and/or mannitol in an amount of about 20 mM to about 40 mM, about 25 mM to about 35 mM, about 26 mM to about 34 mM, about 27 mM to about 33 mM, about 28 mM to about 32 mM, about 29 mM to about 31 mM, about 29.1 mM to about 30.9 mM, about 29.2 mM to about 30.8 mM, about 29.3 mM to about 30.7 mM, about 29.4 mM to about 30.6 mM, about 29.5 mM to about 30.5 mM, about 29.6 mM to about 30.4 mM, about 29.7 mM to about 30.3 mM, about 29.8 mM to about 30.2 mM, about 29.9 mM to about 30.1 mM, or about 30 mM;
KCl in an amount of 0 to about 20 mM, about 5 mM to about 15 mM, about 6 mM to about 14 mM, about 7 mM to about 13 mM, about 8 mM to about 12 mM, about 9 mM to about 11 mM, about 9.1 mM to about 10.9 mM, about 9.2 mM to about 10.8 mM, about 9.3 mM to about 10.7 mM, about 9.4 mM to about 10.6 mM, about 9.5 mM to about 10.5 mM, about 9.6 mM to about 10.4 mM, about 9.7 mM to about 10.3 mM, about 9.8 mM to about 10.2 mM, about 9.9 mM to about 10.1 mM, or about 10 mM; MgCl₂ in an amount of about 10 mM to about 30 mM, about 15 mM to about 25 mM, about 16 mM to about 24 mM, about 17 mM to about 23 mM, about 18 mM to about 22 mM, about 19 mM to about 21 mM, about 19.1 mM to about 20.9 mM, about 19.2 mM to about 20.8 mM, about 19.3 mM to about 20.7 mM, about 19.4 mM to about 20.6 mM, about 19.5 mM to about 20.5 mM, about 19.6 mM to about 20.4 mM, about 19.7 mM to about 20.3 mM, about 19.8 mM to about 20.2 mM, about 19.9 mM to about 20.1 mM, or about 20 mM;
Na₂HPO₄/NaH₂PO₄ in an amount of about 95 mM to about 115 mM, about 100 mM to about 110 mM, about 101 mM to about 109 mM, about 102 mM to about 108 mM, about 103 mM to about 107 mM, about 104 mM to about 106 mM, about 104.1 mM to about 105.9 mM, about 104.2 mM to about 105.8 mM, about 104.3 mM to about 105.7 mM, about 104.4 mM to about 105.6 mM, about 104.5 mM to about 105.5 mM, about 104.6 mM to about 105.4 mM, about 104.7 mM to about 105.3 mM, about 104.8 mM to about 105.2 mM, about 104.9 mM to about 105.1 mM, or about 105 mM; and HEPES in an amount of 0 to about 10 mM; about 1 mM to about 9 mM, about 2 mM to about 8 mM, about 3 mM to about 7 mM, about 4 mM to about 6 mM, about 4.1 mM to about 5.9 mM, about 4.2 mM to about 5.8 mM, about 4.3 mM to about 5.7 mM, about 4.4 mM to about 5.6 mM, about 4.5 mM to about 5.5 mM, about 4.6 mM to about 5.4 mM, about 4.7 mM to about 5.3 mM, about 4.8 mM to about 5.2 mM, about 4.9 mM to about 5.1 mM, or about 5 mM.
   In certain embodiments, the buffer has a pH of from about 6.0 to about 8.0, about 6.1 to about 7.9, about 6.2 to about 7.8, about 6.3 to about 7.7, about 6.4 to about 7.6, about 6.5 to about 7.5, about 6.6 to about 7.4, about 6.7 to about 7.3, about 6.8 to about 7.2, about 6.9 to about 7.1, or about 7.0. In certain embodiments, the buffer has a conductivity of about 13.3 ms/cm to about 15.3 ms/cm, about 13.4 ms/cm to about 15.2 ms/cm, about 13.5 ms/cm to about 15.1 ms/cm, about 13.6 ms/cm to about 15.0 ms/cm, about 13.7 ms/cm to about 14.9 ms/cm, about 13.8 ms/cm to about 14.8 ms/cm, about 13.9 ms/cm to about 14.7 ms/cm, about 14.0 ms/cm to about 14.6 ms/cm, about 14.1 ms/cm to about 14.5 ms/cm, about 14.2 ms/cm to about 14.4 ms/cm, or about 14.3 ms/cm. In certain embodiments, the buffer has an osmolality of about 330 mOsm/kg H₂O to about 350 mOsm/kg H₂O, about 335 mOsm/kg H₂O to about 345 mOsm/kg H₂O, about 336 mOsm/kg H₂O to about 344 mOsm/kg H₂O, about 337 mOsm/kg H₂O to about 343 mOsm/kg H₂O, about 338 mOsm/kg H₂O to about 342 mOsm/kg H₂O, about 339 mOsm/kg H₂O to about 341 mOsm/kg H₂O, about 339.1 mOsm/kg H₂O to about 340.9 mOsm/kg H₂O, about 339.2 mOsm/kg H₂O to about 340.8 mOsm/kg H₂O, about 339.3 mOsm/kg H₂O to about 340.7 mOsm/kg H₂O, about 339.4 mOsm/kg H₂O to about 340.6 mOsm/kg H₂O, about 339.5 mOsm/kg H₂O to about 340.5 mOsm/kg H₂O, about 339.6 mOsm/kg H₂O to about 340.4 mOsm/kg H₂O, about 339.7 mOsm/kg H₂O to about 340.3 mOsm/kg H₂O, about 339.8 mOsm/kg H₂O to about 340.2 mOsm/kg H₂O, about 339.9 mOsm/kg H₂O to about 340.1 mOsm/kg H₂O, or about 340 mOsm/kg H₂O.

In some embodiments, the buffer, consists essentially of, or consists of: water; glucose in an amount of about 30 mM; KCl in an amount of about 10 mM; MgCl₂ in an amount of about 20 mM; Na₂HPO₄/NaH₂PO₄ in an amount of about 105 mM; and HEPES in an amount of about 5 mM. In certain such embodiments, the buffer has a pH of about 7.0, a conductivity of about 14.3 ms/cm, and an osmolality of about 340 mOsm/kg H₂O. In some embodiments, DMSO is specifically excluded from the buffer. In certain embodiments, the buffer comprises, consists essentially of, or consists of: water; glucose in an amount of about 30 mM; KCl in an amount of about 10 mM; MgCl₂ in an amount of about 20 mM; Na₂HPO₄/NaH₂PO₄ in an amount of about 105 mM; HEPES in an amount of about 5 mM; and DMSO in an amount equal to or less than about 2.5% by volume of the total volume of the buffer.
In some embodiments, the buffer comprises, consists essentially of, or consists of: water;
glucose and/or mannitol in an amount of about 26 mM to about 36 mM, about 27 mM to about 35 mM, about 28 mM to about 34 mM, about 29 mM to about 33 mM, about 30 mM to about 32 mM, about 30.1 mM to about 31.9 mM, about 30.2 mM to about 31.8 mM, about 30.3 mM to about 31.7 mM, about 30.4 mM to about 31.6 mM, about 30.5 mM to about 31.5 mM, about 30.6 mM to about 31.4 mM, about 30.7 mM to about 31.3 mM, about 30.8 mM to about 31.2 mM, about 30.9 mM to about 31.1 mM, or about 31 mM;
KCl in an amount of 0 to about 15 mM, 0 to about 10 mM, about 1 mM to about 9 mM, about 2 mM to about 8 mM, about 3 mM to about 7 mM, about 4 mM to about 6 mM, about 4.1 mM to about 5.9 mM, about 4.2 mM to about 5.8 mM, about 4.3 mM to about 5.7 mM, about 4.4 mM to about 5.6 mM, about 4.5 mM to about 5.5 mM, about 4.6 mM to about 5.4 mM, about 4.7 mM to about 5.3 mM, about 4.8 mM to about 5.2 mM, about 4.9 mM to about 5.1 mM, or about 5.0 mM;
MgCl₂ in an amount of about 5 mM to about 25 mM, about 10 mM to about 20 mM, about 11 mM to about 19 mM, about 12 mM to about 18 mM, about 13 mM to about 17 mM, about 14 mM to about 16 mM, about 14.1 mM to about 15.9 mM, about 14.2 mM to about 15.8 mM, about 14.3 mM to about 15.7 mM, about 14.4 mM to about 15.6 mM, about 14.5 mM to about 15.5 mM, about 14.6 mM to about 15.4 mM, 14.7 mM to about 15.3 mM, 14.8 mM to about 15.2 mM, about 14.9 mM to about 15.1 mM, or about 15 mM; and Na₂HPO₄/NaH₂PO₄ in an amount of about 80 mM to about 100 mM, about 85 mM to about 95 mM, about 86 mM to about 94 mM, about 87 mM to about 93 mM, about 88 mM to about 92 mM, about 89 mM to about 91 mM, about 89.1 mM to about 90.9 mM, about 89.2 mM to about 90.8 mM, about 89.3 mM to about 90.7 mM, about 89.4 mM to about 90.6 mM, about 89.5 mM to about 90.5, about 89.6 mM to about 90.4 mM, about 89.7 mM to about 90.3 mM, about 89.8 mM to about 90.2 mM, about 89.9 mM to about 90.1 mM, or about 90 mM.
   In certain embodiments, the buffer has a pH of from about 6.1 to about 8.1, about 6.2 to about 8.0, about 6.3 to about 7.9, about 6.4 to about 7.8, about 6.5 to about 7.7, about 6.6 to about 7.6, about 6.7 to about 7.5, about 6.8 to about 7.4, about 6.9 to about 7.3, about 7.0 to about 7.2, or about 7.1. In certain embodiments, the buffer has a conductivity of about 10.6 ms/cm to about 12.6 ms/cm, about 10.7 ms/cm to about 12.5 ms/cm, about 10.8 ms/cm to about 12.4 ms/cm, about 10.9 ms/cm to about 12.3 ms/cm, about 11.0 ms/cm to about 12.2 ms/cm, about 11.1 ms/cm to about 12.1 ms/cm, about 11.2 ms/cm to about 12.0 ms/cm, about 11.3 ms/cm to about 11.9 ms/cm, about 11.4 ms/cm to about 11.8 ms/cm, about 11.5 ms/cm to about 11.7 ms/cm, about 11.6 ms/cm. In certain embodiments, the buffer has an osmolality of about 270 mOsm/kg H₂O to about 290 mOsm/kg H₂O about 275 mOsm/kg H₂O to about 285 mOsm/kg H₂O, about 276 mOsm/kg H₂O to about 284 mOsm/kg H₂O, about 277 mOsm/kg H₂O to about 283 mOsm/kg H₂O, about 278 mOsm/kg H₂O to about 282 mOsm/kg H₂O, about 279 mOsm/kg H₂O to about 281 mOsm/kg H₂O, about 279.1 mOsm/kg H₂O to about 280.9 mOsm/kg H₂O, about 279.2 mOsm/kg H₂O to about 280.8 mOsm/kg H₂O, about 279.3 mOsm/kg H₂O to about 280.7 mOsm/kg H₂O, about 279.4 mOsm/kg H₂O to about 280.6 mOsm/kg H₂O, about 279.5 mOsm/kg H₂O to about 280.5 mOsm/kg H₂O, about 279.6 mOsm/kg H₂O to about 280.4 mOsm/kg H₂O, about 279.7 mOsm/kg H₂O to about 280.3 mOsm/kg H₂O, about 279.8 mOsm/kg H₂O to about 280.2 mOsm/kg H₂O, about 279.9 mOsm/kg H₂O to about 280.1 mOsm/kg H₂O, or about 280 mOsm/kg H₂O.

In certain embodiments, the buffer comprises, consists essentially of, or consists of: water; glucose in an amount of about 31 mM; KCl in an amount of about 5 mM; and MgCl₂ in an amount of about 15 mM; and Na₂HPO₄/NaH₂PO₄ in an amount of about 90 mM. In certain such embodiments, the buffer has a pH of about 7.1, a conductivity of about 11.6 ms/cm, and an osmolality of about 280 mOsm/kg H₂O. In some embodiments, one or more of HEPES and DMSO is/are specifically excluded from the buffer. In certain embodiments, the buffer comprises, consists essentially of, or consists of: water; glucose in an amount of about 31 mM; KCl in an amount of about 5 mM; and MgCl₂ in an amount of about 15 mM; Na₂HPO₄/NaH₂PO₄ in an amount of about 90 mM; and HEPES in an amount of from about 5 mM to about 10 mM and/or DMSO in an amount equal to or less than about 2.5% by volume of the total volume of the buffer.
In some embodiments, the buffer comprises, consists essentially of, or consists of: water;
glucose and/or mannitol in an amount of about 20 mM to about 40 mM, about 25 mM to about 35 mM, about 26 mM to about 34 mM, about 27 mM to about 33 mM, about 28 mM to about 32 mM, about 29 mM to about 31 mM, about 29.1 mM to about 30.9 mM, about 29.2 mM to about 30.8 mM, about 29.3 mM to about 30.7 mM, about 29.4 mM to about 30.6 mM, about 29.5 mM to about 30.5 mM, about 29.6 mM to about 30.4 mM, about 29.7 mM to about 30.3 mM, about 29.8 mM to about 30.2 mM, about 29.9 mM to about 30.1 mM, or about 30 mM;
KCl in an amount of 0 to about 15 mM, 0 to about 10 mM, about 1 mM to about 9 mM, about 2 mM to about 8 mM, about 3 mM to about 7 mM, about 4 mM to about 6 mM, about 4.1 mM to about 5.9 mM, about 4.2 mM to about 5.8 mM, about 4.3 mM to about 5.7 mM, about 4.4 mM to about 5.6 mM, about 4.5 mM to about 5.5 mM, about 4.6 mM to about 5.4 mM, about 4.7 mM to about 5.3 mM, about 4.8 mM to about 5.2 mM, about 4.9 mM to about 5.1 mM, or about 5.0 mM;
MgCl₂ in an amount of about 5 mM to about 25 mM, about 10 mM to about 20 mM, about 11 mM to about 19 mM, about 12 mM to about 18 mM, about 13 mM to about 17 mM, about 14 mM to about 16 mM, about 14.1 mM to about 15.9 mM, about 14.2 mM to about 15.8 mM, about 14.3 mM to about 15.7 mM, about 14.4 mM to about 15.6 mM, about 14.5 mM to about 15.5 mM, about 14.6 mM to about 15.4 mM, 14.7 mM to about 15.3 mM, 14.8 mM to about 15.2 mM, about 14.9 mM to about 15.1 mM, or about 15 mM; Na₂HPO₄/NaH₂PO₄ in an amount of about 80 mM to about 100 mM, about 85 mM to about 95 mM, about 86 mM to about 94 mM, about 87 mM to about 93 mM, about 88 mM to about 92 mM, about 89 mM to about 91 mM, about 89.1 mM to about 90.9 mM, about 89.2 mM to about 90.8 mM, about 89.3 mM to about 90.7 mM, about 89.4 mM to about 90.6 mM, about 89.5 mM to about 90.5, about 89.6 mM to about 90.4 mM, about 89.7 mM to about 90.3 mM, about 89.8 mM to about 90.2 mM, about 89.9 mM to about 90.1 mM, or about 90 mM; and
HEPES in an amount of 0 to about 20 mM, about 5 mM to about 15 mM, about 6 mM to about 14 mM, about 7 mM to about 13 mM, about 8 mM to about 12 mM, about 9 mM to about 11 mM, about 9.1 mM to about 10.9 mM, about 9.2 mM to about 10.8 mM, about 9.3 mM to about 10.7 mM, about 9.4 mM to about 10.6 mM, about 9.5 mM to about 10.5 mM, 9.6 mM to about 10.4 mM, about 9.7 mM to about 10.3 mM, about 9.8 mM to about 10.2 mM, about 9.9 mM to about 10.1 mM, or about 10 mM.

In certain embodiments, the buffer has a pH of from about 6.1 to about 8.1, about 6.2 to about 8.0, about 6.3 to about 7.9, about 6.4 to about 7.8, about 6.5 to about 7.7, about 6.6 to about 7.6, about 6.7 to about 7.5, about 6.8 to about 7.4, about 6.9 to about 7.3, about 7.0 to about 7.2, or about 7.1. In certain embodiments, the buffer has a conductivity of about 11.8 ms/cm to about 13.8 ms/cm, about 11.9 ms/cm to about 13.7 ms/cm, about 12.0 ms/cm to about 13.6 ms/cm, about 12.1 ms/cm to about 13.5 ms/cm, about 12.2 ms/cm to about 13.4 ms/cm, about 12.3 ms/cm to about 13.3 ms/cm, about 12.4 ms/cm to about 13.2 ms/cm, about 12.5 ms/cm to about 13.1 ms/cm, about 12.6 ms/cm to about 13.0 ms/cm, about 12.7 ms/cm to about 12.9 ms/cm, or about 12.8 ms/cm. In certain embodiments, the buffer has an osmolality of about 282 mOsm/kg H₂O to about 302 mOsm/kg H₂O, 287 mOsm/kg H₂O to about 297 mOsm/kg H₂O, about 288 mOsm/kg H₂O to about 296 mOsm/kg H₂O, about 289 mOsm/kg H₂O to about 295 mOsm/kg H₂O, about 290 mOsm/kg H₂O to about 294 mOsm/kg H₂O, about 291 mOsm/kg H₂O to about 293 mOsm/kg H₂O, about 291.1 mOsm/kg H₂O to about 292.9 mOsm/kg H₂O, about 291.2 mOsm/kg H₂O to about 292.8 mOsm/kg H₂O, about 291.3 mOsm/kg H₂O to about 292.7 mOsm/kg H₂O, about 291.4 mOsm/kg H₂O to about 292.6 mOsm/kg H₂O, about 291.5 mOsm/kg H₂O to about 292.5 mOsm/kg H₂O, about 291.6 mOsm/kg H₂O to about 292.4 mOsm/kg H₂O, about 291.7 mOsm/kg H₂O to about 292.3 mOsm/kg H₂O, about 291.8 mOsm/kg H₂O to about 292.2 mOsm/kg H₂O, about 291.9 mOsm/kg H₂O to about 292.1 mOsm/kg H₂O, or about 292 mOsm/kg H₂O.

In certain embodiments, the buffer comprises, consists essentially of, or consists of: water; glucose in an amount of about 30 mM; KCl in an amount of about 5 mM; and MgCl₂ in an amount of about 15 mM; Na₂HPO₄/NaH₂PO₄ in an amount of about 90 mM; and HEPES in an amount of about 10 mM. In certain such embodiments, the buffer has a pH of about 7.1, a conductivity of about 12.8 ms/cm, and an osmolality of about 292 mOsm/kg H₂O. In some embodiments, DMSO is specifically excluded from the buffer. In certain embodiments, the buffer comprises, consists essentially of, or consists of: water; glucose in an amount of about 30 mM; KCl in an amount of about 5 mM; and MgCl₂ in an amount of about 15 mM; Na₂HPO₄/NaH₂PO₄ in an amount of about 90 mM; HEPES in an amount of about 10 mM; and DMSO in an amount equal to or less than about 2.5% by volume of the total volume of the buffer.

In some embodiments, the buffer is selected from one or more of the exemplary buffers set forth in Tables 2 and 3. In certain embodiments, the buffer is selected from Buffer 1, Buffer 2, or Buffer 3.

In some embodiments, the buffer of the invention is used in conjunction with an UltraPorator™ electroporation apparatus and cartridge (or, cassette); see, WO 2021/096936 (filed Nov. 11, 2020) and U.S. Pre-Grant Publication No. 2021/013837A1 (filed Nov. 11, 2020), each of which is incorporated by reference herein. This apparatus is designed to enable rapid manufacturing for a range of gene and cell therapies. UltraPorator™ is a high-throughput, semi-closed electroporation system for electroporation of large quantities of cells in a single operation. The UltraPorator™ system is an advancement over current electroporation devices by significantly reducing the processing time and contamination risk. For example, UltraPorator may be utilized as a scale-up and commercialization solution for decentralized chimeric antigen receptor (CAR) T-cell manufacturing, such as in the UltraCAR-T™ manufacturing of T-cells reprogrammed to target cancer antigens in vivo.

Buffers of the invention are surprisingly effective in producing high cell transfection efficiencies when electroporation is performed using the buffers in the UltraPorator™ electroporation apparatus and/or cartridge (or, cassette); see, WO 2021/096936 (filed Nov. 11, 2020) and U.S. Pre-Grant Publication No. S221/139837A1.

### Methods and Recombinant Cells Produced Using Those Methods

In another aspect of the invention, a method is provided that utilizes the buffer according to the invention to introduce biologically active material (e.g., DNA or RNA) into cells via electric current (i.e., electroporation). The method comprises: applying an electric current to a suspension comprising isolated eukaryotic cells; a biological material that is exogenous to the cells; and the buffer of the present invention. The suspension is formed by combining cells obtained from a human along with an exogenous biological material into the buffer of the invention. The application of the electric facilitates the introduction of the biological material into the cells. In some embodiments, the eukaryotic cells are human cells. In certain embodiments, the biological material comprises a nucleic acid, a polypeptide, a peptide, and/or a ribonucleoprotein. In certain embodiments, the cells are lymphocytes, for example T cells.

In certain embodiments, the voltage pulse may have a field strength of up to 1 to 10 kV*cm-1 and a duration of 5 to 250 s and a current density of at least 2 A*cm-2. In certain embodiments, the voltage pulse permits the biologically active material (e.g., DNA) to be transfected directly into the cell nucleus of animal and human cells. In certain embodiments, a current flow following the voltage pulse without interruption, having a current density of 2 to 14 A*cm-2, preferably up to 5 A*cm-2, and a duration of 1 to 100 ms, may also be applied.

Using the method according to the invention, the transfection of biologically active material into cells, including into the nucleus of animal cells, may be optimized. In this case, the biologically active material (e.g., nucleic acids, polypeptides, or the like) can be introduced into quiescent or dividing animal cells with a high efficiency.

In some embodiments, the cells are exposed to the buffer for less than 10 minutes. For example, the cells may be exposed to the buffer for less than 9 minutes, less than 8 minutes, less than 7 minutes, less than 6 minutes, less than 5 minutes, less than 4 minutes, less than 3 minutes, less than 2 minutes, or less than 1 minute.

In some embodiments, the method is used to introduce biologically active material into primary human blood cells, pluripotent precursor cells of human blood, as well as primary human fibroblasts and endothelial cells. In some embodiments, the cells are human blood cells, for example immune cells. In certain embodiments, the immune cells are neutrophils, eosinophils, basophils, mast cells, monocytes, macrophages, dendritic cells, natural killer cells, and lymphocytes (B cells and T cells), or some combination thereof. In some embodiments, the lymphocytes are T-cells. In certain embodiments, the cells are obtained from a patient.

In some embodiments, the biological material includes a nucleic acid, peptide, polypeptide, protein, enzyme, RNP, or some combination thereof. In some embodiments, the biological material is heterologous to the cells. In some embodiments, the biological material is partially or fully synthetic.

In some embodiments, the nucleic acid is selected from DNA or RNA. In some embodiments, the DNA may comprise cDNA. In some embodiments, the RNA may comprise mRNA, tRNA, rtRNA, lncRNA, sRNA, or a combination thereof. In some embodiments, the nucleic acid is a recombinant nucleic acid. In some embodiments, the peptide comprises a polypeptide, protein, enzyme, antibody, antibody fragment, or combination thereof. In some embodiments, the peptide is recombinant.

Methods utilizing the buffer of the invention result in desirably high transfection yields, especially as compared to methods utilizing other electroporation buffers. In some embodiments, the transfection yield with a buffer of the invention is at least about 1.1 times that of the transfection yield with a control (prior art) buffer. For example, the transfection yield with a buffer of the invention may be about 1.1, 1.2, 1.3, 1.4, 1.5, 1.6, 1.7, 2.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 times higher than that of a control (prior art) buffer. In some embodiments, the transfection yield with a buffer of the invention may be greater than 5 times than that of a control (prior art) buffer, such as 6, 7, 8, 9, or 10 times higher. In certain embodiments, the transfection yield with a buffer of the invention is 1.35, 1.41, 1.46, 1.97, 1.98, 2.05, 2.12, 2.40, or 2.44 times higher than that of a control (prior art) buffer. In some embodiments, the transfection yield with a buffer of the invention is at least 1.35 times higher than when a control buffer is used.

Methods utilizing the buffer of the invention result in desirably high transfected cell recovery yields, especially as compared to methods utilizing other electroporation buffers. In some embodiments, the transfected cell recovery yield with a buffer of the invention is at least about 1.1 times that of the transfected cell recovery yield with a control (prior art) buffer. For example, the transfected cell recovery yield with a buffer of the invention may be about 1.1, 1.2, 1.3, 1.4, 1.5, 1.6, 1.7, 2.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 higher than that of a control (prior art) buffer. In some embodiments, the transfected cell recovery yield with a buffer of the invention may be greater than 5 times than that of a control (prior art) buffer. In certain embodiments, the transfected cell recovery yield with a buffer of the invention is 1.53, 1.66, 1.72, 1.80, 2.06, 2.17, 2.23, 2.34, or 2.61 times higher than that of a control (prior art) buffer. In some embodiments, the transfected cell recovery yield with a buffer of the invention is at least 1.53 times higher than when a control buffer is used.

The present invention also relates in part to a method of increasing transfection efficiency, the method comprising: combining insolated eukaryotic cells and a biological material that is exogenous to the cells with the buffer of the present invention, thereby forming a suspension; and applying an electric current to the suspension, thereby facilitating the introduction of the biological material into the cells.

The present invention also relates in part to a method of increasing the recovery of transfected cells, the method comprising: combining insolated eukaryotic cells and a biological material that is exogenous to the cells with the buffer of the present invention, thereby forming a suspension; and applying an electric current to the suspension, thereby facilitating the introduction of the biological material into the cells.

In another aspect of the invention, recombinant cells are provided. These cells, produced using the methods described herein, are particularly well suited for diagnostic and/or analytical methods, as well as for the production of biological products for ex-vivo gene therapy, for example immunotherapy and/or CAR-T therapy. In some embodiments, recombinant immune cells are produced using the method of the invention. In some embodiments, the cell is a recombinant human immune cell. In certain embodiments, the cell is a recombinant lymphocyte. In certain embodiments, the cell is a recombinant T-cell. In certain embodiments, the recombinant immune cell is a modified T-cell. In some embodiments, the modified T-cell is a chimeric antigen receptor (CAR) T-cell. In some embodiments, the CAR-T cell is administered to a patient for therapeutic purposes.

The present invention also relates in part to a method of immunotherapy using a recombinant T-cell that has been produced using a method that utilizes the buffer of the present invention.

The present invention also relates in part to a method of immunotherapy using a chimeric antigen receptor (CAR) T-cell that has been produced using a method that utilizes the buffer of the present invention.

The present invention also relates in part to the use of a recombinant T-cell that has been produced using a method that utilizes the buffer of the present invention in the preparation of a medicament for the treatment of a disease or disorder.

The present invention also relates in part to the use of a CAR T-cell that has been produced using a method that utilizes the buffer of the present invention in the preparation of a medicament for the treatment of a disease or disorder.

### Electroporation Apparatuses and Their Methods of Use

In another aspect of the invention, an electroporation apparatus is provided, as well as uses of the apparatus. In some embodiments, the apparatus comprises: one or more chambers configured to store the buffer and cells during an electroporation process; one or more pairs of electrodes configured to generate electric fields within the one or more chambers during the electroporation process, each electric field corresponding to one chamber; and a flow channel configured to transport the cells during a cell collection process after the electroporation process. In some embodiments, the apparatus further comprises: an inlet port; an outlet port; and a flanking flow channel connecting the inlet port and the outlet port to the flow channel.

In some embodiments, the apparatus comprises one chamber, two chambers, three chambers, four chambers, five chambers, six chambers, seven chambers, eight chambers, nine chambers, ten chambers, or ten or more chambers. In certain embodiments, the apparatus utilizes continuous flow or a microfluidic system.

In some embodiments, the electroporation apparatus further comprises a pump for pumping a liquid medium from the flow channel into at least one of the chambers during a collection process, wherein the liquid medium is obtained at the inlet port. In some embodiments, the pump comprises a valve or valves connecting the one or more chambers to the flow channel. In some embodiments, the valve or valves are opened one at a time. In some embodiments, the valve or valves permit only one-directional flow of fluid. In some embodiments, each valve corresponds to one chamber. In some embodiments, each valve corresponding to the chamber valves is a pinch-valve or pinch-type valve. In some embodiments, each of the valves operates using a spring motion, a lever motion, or a piston motion.

In some embodiments, the one or more chambers comprises a given chamber; each electrode of the pair of electrodes is located on opposite sides of the given chamber; and each electrode of the pair of electrodes comprises both an interior portion inside the given chamber and an exterior portion external to the given chamber.

In some embodiments, the electroporation apparatus further comprises: an inlet port; an outlet port; and one or more flanking flow channels connecting the inlet port and the outlet port to the flow channel.

In some embodiments, the electroporation apparatus further comprises: a pump for pumping a liquid medium from the flow channel into at least one of the chambers during a collection process, wherein the liquid medium is obtained at the inlet port.

In some embodiments, the electroporation apparatus further comprises: a surface comprising a one or more openings leading to the one or more chambers; and an airflow channel below the openings and connecting airflow between the chambers.

In some embodiments, the electroporation apparatus further comprises: a vent or air filter connecting the airflow channel to an exterior of the electroporation apparatus.

In some embodiments, the electroporation apparatus further comprises: a seal configured to cover the one or more openings. In some embodiments, each chamber in the electroporation apparatus comprises a shape which narrows toward the respective valve(s). In some embodiments, the electroporation apparatus further comprises a pair of electrodes wherein each electrode of the electrode pair is located on opposite sides of each chamber. The distance between the two electrodes in an electrode pair is referred to as the “gap distance” or “separation distance.” This distance spans the width of the chamber.

In some embodiments, each of the one or more chambers comprises a gap distance of about 0.1 millimeter (mm) to about 20 mm, about 0.5 mm to about 10 mm, about 1 mm to about 7 mm, or about 1 mm to about 4 mm. In some embodiments, the gap distance is about 0.5 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, or 8.0 mm. In some embodiments, a gap distance of about 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, or 4.0 mm. In some embodiments, the gap distance is less than about 4 mm, less than about 3.5 mm, less than about 3.0 mm, less than about 2.5 mm, less than about 2.0 mm, less than about 1.5 mm, or less than about 1.0 mm. In some embodiments, a gap distance of less than about 4.0 mm improves the electroporation performance of the buffer provided herein.

In some embodiments, each electrode of the pair of electrodes of the electroporation apparatus comprises: an interior portion inside the given chamber; and an exterior portion external to the given chamber, wherein each pair of electrodes is configured to connect to an electric circuit. In some embodiments, the interior portion inside the given chamber has an elliptical face and comprises a gold coating.

In some embodiments, each chamber of the electroporation apparatus is configured to store a volume of at least about 50 microliter (μL), at least about 100 μL, at least about 150 at least about L, at least about 200 μL, at least about 250 μL, at least about 300 μL, at least about 350 μL, at least about 400 μL, at least about 450 μL, at least about 150 μL, at least about 500 μL, at least about 550 μL, at least about 600 μL, at least about 650 μL, at least about 700 μL, at least about 750 μL, at least about 800 μL, at least about 850 μL, at least about 900 μL, at least about 950 μL, or at least about 1000 μL (1.0 mL).

In some embodiments, the chambers of the electroporation apparatus, in combination, are configured to store at least about 500 μL, at least about 1.0 milliliter (mL), at least about 1.2 mL, at least about 1.4 mL, at least about 1.6 mL, at least about 1.8 mL, at least about 2.0 mL, at least about 2.2 mL, at least about 2.4 mL, at least about 2.6 mL, at least about 2.8 mL, at least about 3.0 mL, at least about 3.2 mL, at least about 3.4 mL, at least about 3.6 mL, at least about 3.8 mL, at least about 4.0 mL, at least about 4.2 mL, at least about 4.4 mL, at least about 4.6 mL, at least about 4.8 mL, at least about 5.0 mL, at least about 5.2 mL, at least about 5.4 mL, at least about 5.6 mL, at least about 5.8 mL, at least about 6.0 mL, at least about 6.2 mL, at least about 6.4 mL, at least about 6.6 mL, at least about 6.8 mL, or at least about 7.0 mL of cells in liquid suspension for electroporation.

In some embodiments, the cells involved in the electroporation process comprises a population selected from a group consisting of: at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, at least 2×10⁹ cells, at least 3×10⁹ cells, at least 4×10⁹ cells, at least 5×10⁹ cells, at least 6×10⁹ cells, at least 7×10⁹ cells, at least 8×10⁹ cells, at least 9×10⁹ cells, at least 1×10¹⁰ cells, at least 2×10¹⁰ cells, at least 3×10¹⁰ cells, at least 4×10¹⁰ cells, at least 5×10¹⁰ cells, at least 6×10¹⁰ cells, at least 7×10¹⁰ cells, at least 8×10¹⁰ cells, at least 9×10¹⁰ cells, at least 1×10¹¹ cells, at least 2×10¹¹ cells, at least 3×10¹¹ cells, at least 4×10¹¹ cells, at least 5×10¹¹ cells, at least 6×10¹¹ cells, at least 7×10¹¹ cells, at least 8×10¹¹ cells, at least 9×10¹¹ cells, at least 1×10¹² cells, at least 2×10¹² cells, at least 3×10¹² cells, at least 4×10¹² cells, at least 5×10¹² cells, at least 6×10¹² cells, at least 7×10¹² cells, at least 8×10¹² cells, and at least 9×10¹².

In some embodiments, the apparatus of the invention comprises an UltraPorator™ electroporation apparatus and cartridge (see, WO 2021/096936 and U.S. Pre-Grant Publication No. 2021/0139837A1). As noted above, the UltraPorator™ electroporation apparatus is designed to enable rapid manufacturing for a range of gene and cell therapies. The apparatus may be utilized as a scale-up and commercialization solution for decentralized CAR T-cell manufacturing, such as in the UltraCAR-T™ manufacturing of T-cells reprogrammed to target cancer antigens in vivo.

In some embodiments, the apparatus of the invention is used in a method of electroporation, the method comprising: executing an electroporation process by generating an electric field within a chamber using a pair of electrodes, wherein the chamber is configured to store the buffer and cells during the electroporation process; and executing a cell collection process by: opening a valve connected to the chamber; and transporting the buffer and cells to an outlet port using a flow channel connected to the valve, wherein the chamber, the electrode pair, the valve, the outlet port, and the flow channel are each located within an electroporation apparatus.

In some embodiments, the step of executing a cell collection process further comprises: pumping, through use of a pump, a liquid medium from the flow channel into the chamber, wherein the liquid medium is obtained at an inlet port, and wherein the inlet port and the outlet port are connected to the flow channel by a flanking flow channel within the electroporation apparatus.

In some embodiments, the cell collection process further comprises: draining the chamber into the flow channel, wherein pressure within the chamber is maintained via a vent or air filter connected to an air flow channel running between the chamber and another chamber.

In some embodiments, the method of electroporation further comprises: depositing the cells into an opening leading to the chamber holding the buffer; applying a seal to the opening; and connecting the electrode pair to at least one circuit by, for example, inserting the electroporation apparatus into a docking station.

In some embodiments, the method utilizes one or more of the exemplary buffers set forth in Tables 2 and 3. In certain embodiments, the method utilizes Buffer 1, Buffer 2, or Buffer 3.

In some embodiments, the method is performed in an UltraPorator™ electroporation apparatus (see, WO 2021/096936 and U.S. Pre-Grant Publication No. In certain embodiments, the method is performed in an UltraPorator™ electroporation apparatus and utilizes one or more of the exemplary buffers set forth in Tables 2 and 3. In certain embodiments, the method is performed in an UltraPorator™ electroporation apparatus and utilizes Buffer 1, Buffer 2, or Buffer 3 (as set forth in Table 2).

### Electroporation Systems

In another aspect of the invention, a system for electroporation is provided. In some embodiments, the system for electroporation comprises an electroporation apparatus, as described herein, and an electroporation buffer, as described herein. In some embodiments, the electroporation system comprises and UltraPorator™ electroporation apparatus and cartridge (see, WO 2021/096936 and U.S. Pre-Grant Publication No. 2021/0139837A1). As noted above, the UltraPorator™ electroporation apparatus is designed to enable rapid manufacturing for a range of gene and cell therapies. The device may be utilized as a scale-up and commercialization solution for decentralized CAR T-cell manufacturing, such as in the UltraCAR-T™ manufacturing of T-cells reprogrammed to target cancer antigens in vivo.

In some embodiments, the system for electroporation further comprises a buffer is from one or more of the exemplary buffers set forth in Tables 2 and 3. In certain embodiments, the system for electroporation comprises a buffer selected from Buffer 1, Buffer 2, or Buffer 3. It has been found that systems comprising an UltraPorator™ device and one of Buffers 1, 2, or 3 result in surprisingly high cell transfection efficiencies, as compared to systems comprising an UltraPorator™ device and a control buffer.

### Kits

In another aspect of the invention, a kit is provided. In some embodiments, the kit comprises: a buffer of the present invention; and a dropper, pipette, or cuvette. The kit may include any of the buffers as described herein. In some embodiments, the kit comprises one or more of the exemplary buffers set forth in Tables 2 and 3. In certain embodiments, the kit comprises a buffer selected from Buffer 1, Buffer 2, or Buffer 3. In some embodiments, the kit includes one or more containers filled with a buffer according to the invention and other suitable reagents and/or devices. For example, the kit may additionally comprise a vector comprising a nucleic acid of interest. In some embodiments, the kit may include a dropper, pipette, and/or cuvette. In some embodiments, the buffer may be packaged in aliquoted containers or as a stock solution.

In some embodiments, the kit further comprises packaging to safely transport the buffer and any additional reagents and/or devices. In some embodiments, the kit includes information about the contents of the buffer and any additional reagents. Further, the kit may comprise written materials, for example a user manual or answers to frequently asked questions.

### EXAMPLES

The following non-limiting examples are provided to further illustrate the described embodiments and not to limit the scope of the invention.

### Example 1: Preparation of a Sodium Phosphate Buffering Agent

To investigate the impact on the ratio of monobasic phosphate to dibasic phosphate, alternative buffering agent of different ratios of 0.2 M NaH₂PO₄—H₂O were combined with 0.2 M Na₂HPO₄ and tested to evaluate the ratio's impact on the electroporation (EP) buffer's performance. Table 1 provides the ratios of Na₂HPO₄ to NaH₂PO₄ and their corresponding pHs. A first 0.2 M stock solution of NaH₂PO₄—H₂O (27.6 g/L) and a second 0.2 M stock solution of Na₂HPO₄ (28.4 g/L) were prepared. The first stock solution was combined with the second stock solution as provided in Table 1. The resulting mixture was then further diluted to a total volume of 200 mL to produce a 0.1 M phosphate buffer of the required pH at room temperature.

TABLE 1
Differing Amounts of Monobasic and Dibasic
Phosphate Used as a Buffering Agent

| 0.2M NaH₂PO₄ | 0.2M Na₂HPO₄ | |
|---|---|---|
| (mL) | (mL) | pH |
| 92.0 | 8.0 | 5.8 |
| 90.0 | 10.0 | 5.9 |
| 87.7 | 12.3 | 6.0 |
| 85.5 | 15.0 | 6.1 |
| 81.5 | 19.5 | 6.2 |
| 77.5 | 22.5 | 6.3 |
| 73.5 | 26.5 | 6.4 |
| 68.5 | 31.5 | 6.5 |
| 62.5 | 37.5 | 6.6 |
| 56.5 | 43.5 | 6.7 |
| 51.0 | 49.0 | 6.8 |
| 45.0 | 55.0 | 6.9 |
| 39.0 | 61.0 | 7.0 |
| 33.0 | 67.0 | 7.1 |
| 28.0 | 72.0 | 7.2 |
| 23.0 | 77.0 | 7.3 |
| 19.0 | 81.0 | 7.4 |
| 16.0 | 84.0 | 7.5 |
| 13.0 | 87.0 | 7.6 |
| 10.5 | 89.5 | 7.7 |
| 8.5 | 91.5 | 7.8 |

The various sodium phosphate buffering agents as described above were evaluated in buffers at an amount of about 50 mM to about 160 mM. The results demonstrate that performance was not negatively affected when the pH of the sodium phosphate buffering agent is in the range of about 6.85 pH to about 7.7 pH.

### Example 2: Preparation of Exemplary Buffers (1-37)

Tables 2 and 3 show the composition of exemplary buffers that were prepared. Buffers 1 through 20 comprise glucose (Table 2), whereas Buffers 21 through 37 comprise mannitol (Table 3). Of these buffers, three (referred to herein as Buffers 1, 2, and 3) were subsequently tested against a control buffer (Mirus Bio™ Ingenio™ electroporation solution, Catalog No. MIR-50117; Mirus Bio LLC, Madison, Wis., USA) (“Control 1”). See Example 3.

TABLE 2
Buffers 1 through 20—Buffering Agents and Glucose

| | | | Na₂HPO₄/ | | | |
|---|---|---|---|---|---|---|
| Sample | Glucose | HEPES | NaH₂PO₄ | KCl | MgCl₂ | DMSO |
| No. | (mM) | (mM) | (mM) | (mM) | (mM) | (%) |
| 1 | 30 | 5 | 105 | 10 | 20 | 0 |
| 2 | 31 | 0 | 90 | 5 | 15 | 0 |
| 3 | 30 | 10 | 90 | 5 | 15 | 0 |
| 4 | 25 | 10 | 120 | 15 | 25 | 0 |
| 5 | 30 | 25 | 50 | 2 | 10.5 | 5 |
| 6 | 0 | 5 | 160 | 10 | 10.5 | 0 |
| 7 | 0 | 5 | 160 | 2 | 20 | 5 |
| 8 | 15 | 25 | 160 | 10 | 20 | 5 |
| 9 | 30 | 5 | 160 | 2 | 1 | 2.5 |
| 10 | 15 | 15 | 105 | 6 | 10.5 | 2.5 |
| 11 | 30 | 25 | 50 | 10 | 1 | 0 |
| 12 | 30 | 5 | 50 | 6 | 20 | 5 |
| 13 | 30 | 15 | 160 | 10 | 1 | 5 |
| 14 | 15 | 5 | 50 | 2 | 1 | 0 |
| 15 | 0 | 5 | 50 | 10 | 1 | 5 |
| 16 | 0 | 25 | 50 | 10 | 20 | 2.5 |
| 17 | 30 | 25 | 160 | 2 | 20 | 0 |
| 18 | 0 | 15 | 50 | 2 | 20 | 0 |
| 19 | 0 | 25 | 160 | 6 | 1 | 0 |
| 20 | 0 | 25 | 105 | 2 | 1 | 5 |

TABLE 3
Buffers 21 through 37—Buffering Agents and Mannitol

| | | | Na₂HPO₄/ | | | |
|---|---|---|---|---|---|---|
| Sample | Mannitol | HEPES | NaH₂PO₄ | KCl | MgCl₂ | DMSO |
| No. | (mM) | (mM) | (mM) | (mM) | (mM) | (%) |
| 21 | 5 | 25 | 160 | 6 | 1 | 0 |
| 22 | 150 | 25 | 50 | 2 | 10.5 | 5 |
| 23 | 5 | 15 | 50 | 2 | 20 | 0 |
| 24 | 150 | 25 | 50 | 10 | 1 | 0 |
| 25 | 5 | 25 | 105 | 2 | 1 | 5 |
| 26 | 77.5 | 5 | 50 | 2 | 1 | 0 |
| 27 | 150 | 5 | 160 | 2 | 1 | 2.5 |
| 28 | 150 | 15 | 160 | 10 | 1 | 5 |
| 29 | 5 | 5 | 50 | 10 | 1 | 5 |
| 30 | 150 | 25 | 160 | 2 | 20 | 0 |
| 31 | 150 | 5 | 105 | 10 | 20 | 0 |
| 32 | 77.5 | 15 | 105 | 6 | 10.5 | 2.5 |
| 33 | 77.5 | 25 | 160 | 10 | 20 | 5 |
| 34 | 150 | 5 | 50 | 6 | 20 | 5 |
| 35 | 5 | 25 | 50 | 10 | 20 | 2.5 |
| 36 | 5 | 5 | 160 | 10 | 10.5 | 0 |
| 37 | 5 | 5 | 160 | 2 | 20 | 5 |

### Example 3: Properties of Buffers 1, 2, and 3

Table 4 provides the composition, pH, conductivity, and osmolality of three exemplary buffers—Buffers 1, 2, and 3 prepared in Example 2—as well as a control buffer (Mirus Bio™ Ingenio™ electroporation solution, Catalog No. MIR-50117; Mirus Bio LLC, Madison, Wis., USA) (“Control 1”).

TABLE 4
Composition, pH, Conductivity, and Osmolality of Buffers 1, 2, and 3
Compared to Control 1

| | | | Na2HPO4/ | | | | | osm |
|---|---|---|---|---|---|---|---|---|
| Sample | Glucose | HEPES | NaH2PO4 | KCl | MgCl2 | | Conductivity | (mOsm/ |
| No. | (mM) | (mM) | (mM) | (mM) | (mM) | pH | (ms/cm) | kg H₂O) |
| 1 | 30 | 5 | 105 | 10 | 20 | 7.0 | 14.3 | 340 |
| 2 | 31 | 0 | 90 | 5 | 15 | 7.1 | 11.6 | 280 |
| 3 | 30 | 10 | 90 | 5 | 15 | 7.1 | 12.8 | 292 |
| Control 1 | X | X | | | | 7.3 | 16.9 | 575 |

### Example 4: Transfection of a CAR Construct into Donors' Cells Using Buffers 1, 2, and 3

To study the transfection and survival rates of cells during electroporation as a function of the buffer capacity, primary human lymphocytes were obtained from three donors. Standard apheresis leukopak and PBMC enrichment was used to isolate the lymphocytes. (See, e.g., A. Garcia et al., “Leukopak PBMC Sample Processing for Preparing Quality Control Material to Support Proficiency Testing Programs,” J. Immunol. Methods. 409: 99-106 (July 2014); D. M. Ward, “Conventional Apheresis Therapies: A Review,” J. Clin. Apheresis 26: 230-238; L. Trajman, “Leukopak 101: A Brief Review of Apheresis,” each of which is hereby incorporated by reference.)

Approximately equal numbers of lymphocytes were suspended in Buffers 1, 2, 3, and the control buffer, and then transfected under substantially identical electroporation conditions with a nucleic acid vector encoding a first chimeric antigen receptor (CAR1). The composition of Buffers 1, 2, and 3 are set forth in Table 2. The control buffer used was Mirus Bio™ Ingenio™ electroporation solution (Catalog No. MIR-50117; Mirus Bio LLC, Madison, Wis., USA) (“Control 1”). An UltraPorator™ device (see WO 2021/096936 and U.S. Pre-Grant Publication No. 2021/0139837A1) was used to perform the electroporation. Immediately after electroporation, the samples were transferred to recovery media flasks.

The results of the experiment are shown in Table 5. These results were obtained using standard analysis techniques, including flow cytometry, viability analysis, and cell counting. (See, e.g., G. De Libero, “T Cell Protocols”, Springer Protocols, 2d ed. (2009), incorporated herein by reference.) “Viability” provides the percentage of viable cells prior to electroporation, “Cell Recovery” is the percentage of cells that survived post-electroporation, “Transfection” is the percentage of cells that were transfected with the nucleic acid, and “Transfected Cell Yield” is the percentage of cells that recovered and contain transfected biological material.

TABLE 5
Results Showing Performance Differences of the Three Exemplary
Buffers and a Control Buffer in an UltraPorator ™ Device

| | | | | Cell | Trans- | Transfected |
|---|---|---|---|---|---|---|
| CAR | | | Viability | Recovery | fection | Cell Yield |
| Construct | Donor | Buffer | % | % | % | % |
| CAR1 | 1 | 1 | 84.1 | 55.7 | 42.8 | 23.6 |
| | | 2 | 80.0 | 53.0 | 41.4 | 21.8 |
| | | 3 | 80.0 | 54.7 | 51.0 | 27.7 |
| | | Control 1 | 84.8 | 51.1 | 20.9 | 10.6 |
| CAR1 | 2 | 1 | 82.7 | 46.8 | 52.1 | 24.3 |
| | | 2 | 81.8 | 48.9 | 56.3 | 27.4 |
| | | 3 | 83.0 | 48.5 | 54.5 | 26.4 |
| | | Control 1 | 76.6 | 41.2 | 38.6 | 15.9 |
| CAR1 | 3 | 1 | 77.1 | 40.9 | 38.5 | 15.5 |
| | | 2 | 81.7 | 43.5 | 46.8 | 20.1 |
| | | 3 | 83.8 | 45.6 | 41.4 | 18.7 |
| | | Control 1 | 81.3 | 44.6 | 19.5 | 8.6 |

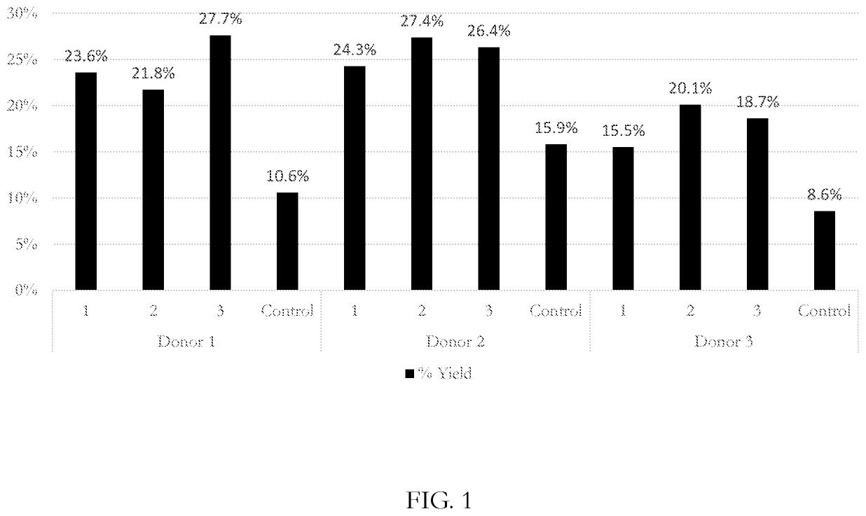

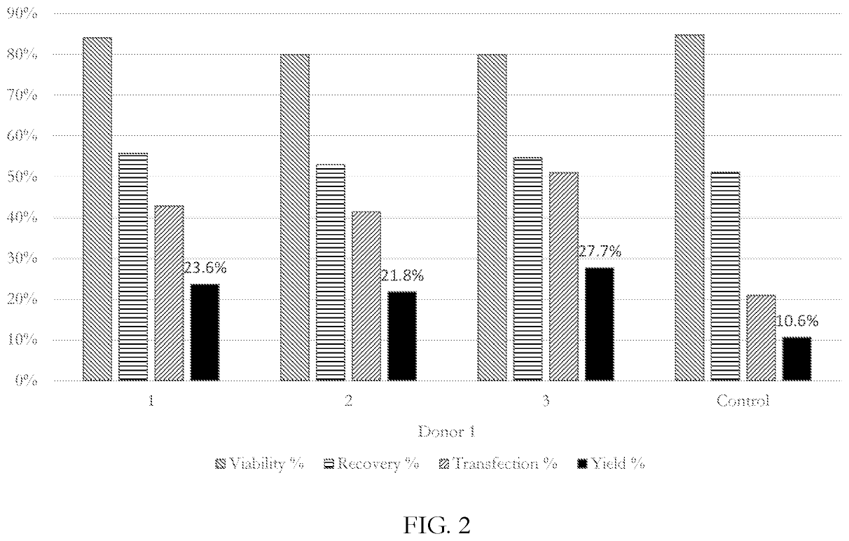

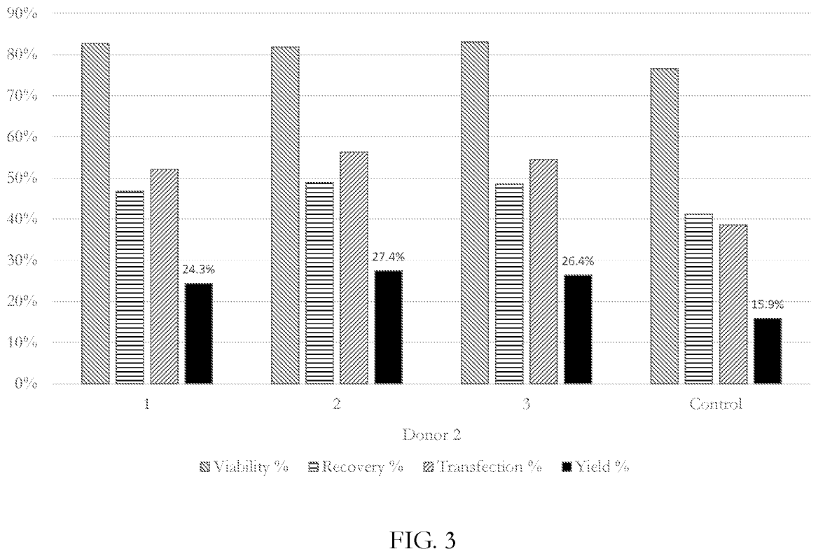

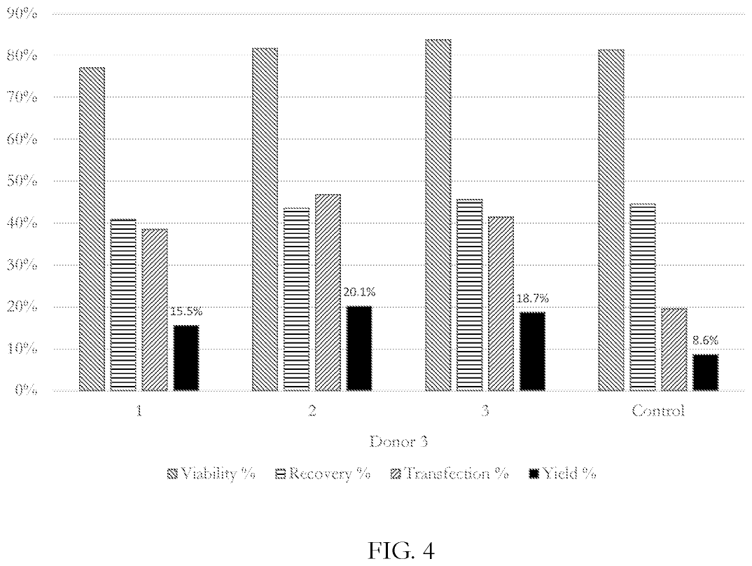

As shown in Table 5, the three buffers (Buffers 1, 2, and 3) had a significantly higher percent yield than the control buffer. Referring to FIG. 1 and FIG. 2, for example, in the lymphocytes taken from Donor 1, Buffers 1, 2, and 3 resulted in transfection yields 2.05, 1.98, and 2.44 times higher than the control buffer, respectively, and corresponding transfected cell recovery yields of 2.23, 2.06, and 2.61 times higher than the control buffer, respectively. Referring to FIG. 1 and FIG. 3, in the lymphocytes taken from Donor 2, Buffers 1, 2, and 3 resulted in transfection yields 1.35, 1.46, and 1.41 times higher than the control buffer, respectively, and corresponding transfected cell recovery yields of 1.53, 1.72, and 1.66 times higher than the control buffer, respectively. Referring to FIG. 1 and FIG. 4, in the lymphocytes taken from Donor 3, Buffers 1, 2, and 3 resulted in transfection yields 1.97, 2.40, and 2.12 times higher than the control buffer, respectively, and corresponding transfected cell recovery yields of 1.80, 2.34, and 2.17 times higher than the control buffer, respectively.

### Example 5: Transfection of a CAR Construct into Donors' Cells Using Buffers 1, 2, and 3

As in Example 4, approximately equal numbers of lymphocytes were suspended in Buffers 1, 2, 3, and the control buffer. The composition of Buffers 1, 2, and 3 are set forth in Table 2, and the preparation of the sodium phosphate buffering agent is set forth in Example 2. The control buffer used was Mirus Bio™ Ingenio™ electroporation solution (Catalog No. MIR-50117; Mirus Bio LLC, Madison, Wis., USA) (“Control 1”).

The suspended lymphocytes were then transfected under substantially identical electroporation conditions with a nucleic acid vector encoding a second chimeric antigen receptor construct (CAR2). An UltraPorator™ device (see WO 2021/096936 and U.S. Pre-Grant Publication No. 2021/0139837A1) was used to perform the electroporation. Immediately after electroporation, the samples were transferred to recovery media flasks.

The results of the experiment, shown in Table 6, were obtained using standard analysis techniques, including flow cytometry and cell counting, as provided in Example 4.

TABLE 6
Viability, Cell Recovery, Transfection and Transfected Cell Yields
of a CAR Construct Using Buffers 1-3 in an UltraPorator ™ Device

| | | | | Cell | Trans- | Transfected |
|---|---|---|---|---|---|---|
| CAR | | | Viability | Recovery | fection | Cell Yield |
| Construct | Donor | Buffer | % | % | % | % |
| CAR2 | 1 | 1 | 84.8 | 50.0 | 36.6 | 17.8 |
| | | 2 | 82.0 | 48.0 | 39.5 | 18.6 |
| | | 3 | 79.1 | 37.4 | 38.1 | 14.0 |
| | | Control 1 | 83.6 | 45.8 | 16.4 | 7.4 |
| | 2 | 1 | 83.8 | 48.2 | 34.8 | 16.3 |
| | | 2 | 86.3 | 47.0 | 48.2 | 22.2 |
| | | 3 | 84.8 | 43.0 | 46.5 | 19.5 |
| | | Control 1 | 83.0 | 42.6 | 28.6 | 11.9 |

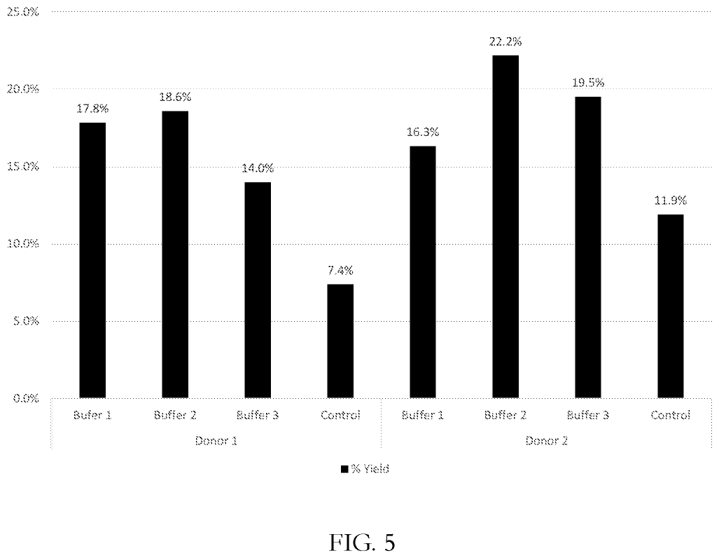

As shown in Table 6, the percentage of recovered lymphocytes successfully transfected with the CAR2 construct was significantly higher using each of Buffers 1, 2, and 3 than it was using the control buffer. Specifically, and referring to FIG. 5, Buffers 1, 2, and 3 resulted in transfected cell yields (of lymphocytes taken from Donor 1) 2.41, 2.51, and 1.89 times higher than the control buffer, respectively. Similarly, Buffers 1, 2, and 3 resulted in transfected cell yields (of lymphocytes taken from Donor 2) 1.37, 1.87, and 1.64 times higher than the control buffer, respectively.

### Example 6: Transfection and Transfected Cell Yields in an UltraPorator™ Device

Primary human lymphocyte cells may be taken from two donors and electroporated in an UltraPorator™ device for evaluation of transfection efficiency (Transfection %) and survival rates (Transfected Cell Yield (%)). Approximately equal numbers of lymphocytes may be suspended in Buffers 1, 2, 3, or in a commercially available buffer, such as, for example, one of the following six commercially available buffer solutions: a) Control Buffer 2: Bio-Rad Gene Pulser® electroporation buffer (Catalog No. 165-2676, Hercules, Calif., USA); b) Control Buffer 3: Neon® Transfection System Buffer (Catalog No. MPK-10025, USA); c) Control Buffer 4: Celetrix® electroporation buffer (Catalog No. 1207, Manassas, Va. 20109 U.S.); d) Control Buffer 5: BTXpress® high performance electroporation solution (Catalog No. 45-0803, Holliston, Mass. 01746); e) Control Buffer 6: Miltenyi Biotec CliniMACS® electroporation buffer (Catalog No. 170-076-625, San Jose, Calif. 95134); and f) Control Buffer 7: Cole-Parmer Eppendorf electroporation buffer for eukaryotic cells (Mfr No. 940002001, Item No. EW-36205-60, Vernon Hills, Ill. 60061).

The cells are transfected under substantially identical electroporation conditions with the same nucleic acid vector comprising a CAR construct, such as CAR1 or CAR2. Any known electroporation device may be used to carry out the transfection, such as, for example, a Bio-Rad Gene Pulser®, Neon® Transfection System, Celetrix® Electroporator, NepaGene® Electroporator, Bulldog Bio® High Voltage Electroporator, CytoFlex® Electroporator, CliniMACS® Electroporator, Eppendorf® Electroporator, or any other known or commercially available electroporation device, including those described in the literature. See, e.g., J. Gehl, “Electroporation: Theory and methods, perspectives for drug delivery, gene therapy and research.” Acta Physiol. Scand., 177:437-447 (2003); M. S. Venslauskas, et al., “Mechanisms of transfer of bioactive molecules through the cell membrane by electroporation,” Eur. Biophys. J. Biophys., 44:277-289 (2015); J. Shi, at al., “A Review on Electroporation-Based Intracellular Delivery,” Molecules, 23(11):3044 (2018); M. B. Fox, et al., “Electroporation of cells in microfluidic devices: a review,” Analytical & Bioanalytical Chem., 385:474 (2006); S. Movahed et al., “Microfluidics cell electroporation,” Microfluidics and Nanofluidics, 10:703-734 (2011); C. A. Lissandrello, et al., “High-throughput continuous-flow microfluidic electroporation of mRNA into primary human T cells for applications in cellular therapy manufacturing,” Sci. Reports, 10: 18045 (2020), J. J. Sherba, et. Al, “The effects of electroporation buffer composition on cell viability and electro-transfection efficiency,” Sci Rep, 10:3053 (2020), each of which is hereby incorporated by reference. Additionally, an UltraPorator™ device (see WO 2021/096936 and U.S. Pre-Grant Publication No. 2021/0139837A1) may be used to perform the electroporation. Immediately after electroporation in buffer solution, the samples may be transferred to recovery media.

Table 7 sets forth expected transfection efficiencies and transfection cell yields which may result from these experiments when performed in an UltraPorator™ device, as may be obtained using standard analysis techniques, including flow cytometry and cell counting, as provided in Example 4.

TABLE 7
Expected Results of Sample Buffers 1, 2, and 3 as Compared to Control
Buffers 2 thru 7 in an UltraPorator ™ Device

| CAR | | Transfection | Transfected Cell |
|---|---|---|---|
| Construct | Buffer | % | Yield (%) |
| CAR1 | 1 | 52.1 | 24.3 |
| | 2 | 56.3 | 27.4 |
| | 3 | 54.5 | 26.4 |
| | Control 2 | <50.0 | <23.0 |
| | Control 3 | <50.0 | <23.0 |
| | Control 4 | <50.0 | <23.0 |
| | Control 5 | <50.0 | <23.0 |
| | Control 6 | <50.0 | <23.0 |
| | Control 7 | <50.0 | <23.0 |
| CAR2 | 1 | 34.8 | 16.3 |
| | 2 | 48.2 | 22.2 |
| | 3 | 46.5 | 19.5 |
| | Control 2 | <33.0 | <16.0 |
| | Control 3 | <33.0 | <16.0 |
| | Control 4 | <33.0 | <16.0 |
| | Control 5 | <33.0 | <16.0 |
| | Control 6 | <33.0 | <16.0 |
| | Control 7 | <33.0 | <16.0 |

As shown in Table 7, Buffers 1, 2, and 3 are each expected to have a statistically significantly higher transfected yield and transfection cell yield percentage as compared to Control Buffers 2 thru 7. The transfected yield and transfection cell yield percentages may range from anywhere between 5% higher (as compared to a control buffer) to over 50% higher.

### REFERENCES

G. De Libero, “T Cell Protocols”, Springer Protocols, 2d ed. (2009).
A. Garcia et al., “Leukopak PBMC Sample Processing for Preparing Quality Control Material to Support Proficiency Testing Programs,” J. Immunol. Methods. 409: 99-106 (July 2014).
M. B. Fox, et al., “Electroporation of cells in microfluidic devices: a review,” Analytical & Bioanalytical Chem., 385:474 (2006).
J. Gehl, “Electroporation: Theory and methods, perspectives for drug delivery, gene therapy and research.” Acta Physiol. Scand., 177:437-447 (2003).
C. A. Lissandrello, et al., “High-throughput continuous-flow microfluidic electroporation of mRNA into primary human T cells for applications in cellular therapy manufacturing,” Sci. Reports, 10: 18045 (2020).
S. Movahed et al., “Microfluidics cell electroporation,” Microfluidics and Nanofluidics, 10:703-734 (2011).
J. J. Sherba, et. Al, “The effects of electroporation buffer composition on cell viability and electro-transfection efficiency,” Sci Rep, 10:3053 (2020).
J. Shi, at al., “A Review on Electroporation-Based Intracellular Delivery,” Molecules, 23(11):3044 (2018).
L. Trajman, “Leukopak 101: A Brief Review of Apheresis” available at
M. S. Venslauskas, et al., “Mechanisms of transfer of bioactive molecules through the cell membrane by electroporation,” Eur. Biophys. J. Biophys., 44:277-289 (2015).
D. M. Ward, “Conventional Apheresis Therapies: A Review,” J. Clin. Apheresis 26: 230-238.
PCT/US2020/059984, filed Nov. 11, 2020, by Shuyuan Zhang et. al, published as WO 2021/096936, entitled, “Electroporation apparatus and method.”
U.S. Pre-Grant Publication No. 2021/0139837A1, filed Nov. 11, 2020.

We claim:

1. A buffer comprising: a solvent; a sugar; a chloride salt; and a buffering agent.

2. The buffer of claim 1, wherein: the solvent is water; the sugar is glucose or mannitol; the chloride salt is potassium chloride (KCl) or magnesium chloride (MgCl₂); and the buffering agent is sodium phosphate, 4-(2-hydroxyethyl)-1-piperazineethanesulfonic acid (HEPES) and/or dimethyl sulfoxide (DMSO).

3. The buffer of claim 1, consisting essentially of: water; glucose or mannitol; KCl; MgCl₂; and sodium phosphate.

4. The buffer of claim 1, comprising glucose or mannitol in an amount of from about 10 mM to about 50 mM.

5. The buffer of claim 1, comprising KCl in an amount of from about 1 mM to about 30 mM.

6. The buffer of claim 1, comprising MgCl₂ in an amount of from about 5 mM to about 50 mM.

7. The buffer of claim 1, comprising sodium phosphate in an amount of from about 50 mM to about 160 mM.

8. The buffer of claim 1, comprising HEPES in an amount of from about 1 mM to about 30 mM.

9. The buffer of claim 1, comprising DMSO in an amount of from about 0% to about 2.5% by volume of the total buffer volume.

10. The buffer of claim 1, comprising: water; glucose or mannitol in an amount of from about 25 mM to about 35 mM; KCl in an amount of from about 5 mM to about 15 mM; MgCl₂ in an amount of from about 15 mM to about 25 mM; and sodium phosphate in an amount of from about 90 mM to about 120 mM.

11. The buffer of claim 10, consisting essentially of: water; glucose or mannitol in an amount of from about 25 mM to about 35 mM; KCl in an amount of from about 5 mM to about 15 mM; MgCl₂ in an amount of from about 15 mM to about 25 mM; and sodium phosphate in an amount of from about 90 mM to about 120 mM.

12. The buffer of claim 10, consisting essentially of: water; glucose or mannitol in an amount of from about 25 mM to about 35 mM; KCl in an amount of from about 5 mM to about 15 mM; MgCl₂ in an amount of from about 15 mM to about 25 mM; sodium phosphate in an amount of from about 90 mM to about 120 mM; and HEPES in an amount of from about 5 mM to about 10 mM and/or DMSO in an amount equal to or less than about 2.5% of by volume of the total volume of the buffer.

13. A method of electroporation, the method comprising applying an electric current to a suspension comprising: isolated eukaryotic cells; a biological material that is exogenous to the cells; and the buffer of claim 1.

14. The method of claim 13, wherein the eukaryotic cells are human cells.

15. The method of claim 13, wherein the biological material comprises a nucleic acid, a polypeptide, a peptide, and/or a ribonucleoprotein.

16. A recombinant cell produced using the method of claim 13.

17. The recombinant cell of claim 16, wherein the cell is a recombinant T-cell.

18. A method of immunotherapy or CAR-T therapy using the recombinant T-cell of claim 17.

19. An electroporation apparatus comprising: one or more chambers; one or more pairs of electrodes configured to generate electric fields within the one or more chambers, wherein each electric field corresponds to one chamber; and a flow channel.

20. A method for electroporation comprising utilizing the electroporation apparatus of claim 19.
